(19) United States
(12) Patent Application Publication
(10) Pub. No.: US 2024/0057652 A1
(43) Pub. Date: Feb. 22, 2024

(54) FOOD COMPOSITIONS COMPRISING METHYLOCOCCUS CAPSULATUS PROTEIN ISOLATE

(71) Applicant: Calysta, Inc., (US)

(72) Inventors: Chien-Seng HWANG, San Mateo, CA (US); Lisa Marie NEWMAN, San Jose, CA (US); Warren KWAN, San Mateo, CA (US); Lori J. GIVER, Sunnyvale, CA (US); Celine SCHIFF-DEB, San Mateo, CA (US)

(21) Appl. No.: 17/766,719

(22) PCT Filed: Oct. 7, 2020
(86) PCT No.: PCT/US2020/054507

§ 371 Date: Apr. 5, 2022

Related U.S. Application Data
(60) Provisional application No. 62/911,970, filed on Oct. 7, 2019.

(51) Int. Cl. A23L 33/195; A21D 2/26; A23C 11/10; A23G 9/38; A23J 1/00; A23J 3/20; A23J 3/22; C12N 1/20

(52) U.S. Cl. CPC A23L 33/195; A21D 2/267; A23C 11/10; A23G 9/38; A23J 1/008; A23J 3/20; A23J 3/227; C12N 1/20

(57) ABSTRACT

The present disclosure provides food compositions for humans comprising a Methylococcus capsulatus protein isolate or whole cell product, wherein the Methylococcus capsulatus protein isolate or whole cell product is composed of at least 70% crude protein; has a protein digestibility corrected amino acid score (PDCAAS) of at least 0.9; and has a δ¹³C of about −70‰ to about −30‰. Methylococcus capsulatus protein isolates or whole cell products provided herein are useful for the manufacture of meat substitutes as well as other food compositions, such as protein bars, nutritional beverages, protein supplement powders, frozen non-dairy desserts, and baked goods.

### FIELD OF THE DISCLOSURE

The present disclosure relates to food compositions for human consumption comprising Methylococcus capsulatus protein isolate.

### BACKGROUND

By 2050, the world's population is estimated to reach 9.5 billion. Feeding a world population of over 9 billion people in 2050 would require raising overall food production, with demand for animal-derived protein projected to double (Henchion et al. 2017, Foods 6:53). However, animal-derived protein raises concerns for sustainability and food security. Animal-based food raises environmental concerns due to: (i) the higher levels of greenhouse gases produced compared to plant-based foods; (ii) high impact on land and water use; and (iii) loss of biodiversity due to conversion of forests, wetlands, and natural grasslands to agricultural use. High consumption of meat-based proteins containing high levels of saturated fatty acids raises health concerns. Production of animal-based proteins also raises ethical concerns regarding industrial livestock production and their slaughter.

Currently, plant-based protein sources make up the majority or the global protein supply (57%). However, plant-based protein sources have suboptimal amino acid composition, often lacking one or more amino acids in sufficient quantities to meet human nutrition requirements (Henchion et al., 2017, Foods 6:53). While plant-based proteins may have more favorable land use and greenhouse gas emission compared to animal-based proteins, they are still subject of environmental concerns, which include high land and water use, soil degradation, and pesticide pollution. Moreover, there are seasonability and scalability issues relating to plant-protein production and taste and functional limitations to use of plant-proteins in certain food compositions (e.g., meat substitutes). Thus, there is a need for new protein sources for human consumption that overcome the environmental, ethical, health, nutritional, concerns of animal- and plant-based proteins while providing scalable and consistent production. The present disclosure meets such needs, and further provides other related advantages.

### SUMMARY

The present disclosure provides food compositions for human consumption comprising Methylococcus capsulatus protein isolate or whole cell product.

In one aspect, the present disclosure provides a food composition for humans comprising Methylococcus capsulatus protein isolate, wherein the Methylococcus capsulatus protein isolate: (a) is composed of more than 70% by weight crude protein; (b) has a protein digestibility corrected amino acid score (PDCAAS) of at least 0.9; and (c) has a δ¹³C of about −70‰ to about −30‰. In further embodiments, the Methylococcus capsulatus protein isolate is composed of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% by weight protein. In yet further embodiments, the Methylococcus capsulatus protein isolate has a δ¹³C of about −60‰ to about −40‰.

In a further embodiment, the Methylococcus capsulatus protein isolate has: (a) less than 6% by weight nucleic acids; (b) less than 7% by weight ash; (c) less than 10% by weight crude fat; (d) at least 0.05 mg heme/g protein isolate; or any combination thereof.

In a further embodiment, the Methylococcus capsulatus is not genetically modified.

In a further embodiment, the Methylococcus capsulatus is Methylococcus capsulatus (Bath), Methylococcus capsulatus (Texas), or Methylococcus capsulatus (Aberdeen).

In a further embodiment, the Methylococcus capsulatus protein isolate is derived from Methylococcus capsulatus grown on a carbon feedstock comprising methane or methanol. In yet further embodiments, the carbon feedstock comprising methane is natural gas or unconventional natural gas.

In a further embodiment, the Methylococcus capsulatus protein isolate is derived from Methylococcus capsulatus grown cultured under low copper conditions. In yet further embodiments, the Methylococcus capsulatus protein isolate is derived from Methylococcus capsulatus cultured in at most 100 mg copper/kg dry cell weight (DCW). In yet further embodiments, the Methylococcus capsulatus protein isolate is derived from Methylococcus capsulatus cultured in 1 to 100, from 1 to 10, from 10 to 20, from 20 to 30, from 30 to 40, from 40 to 50, from 50 to 60, from 60 to 70, from 70 to 80, from 80 to 90, from 90 to 100, from 1 to 90, from 1 to 80, from 1 to 70, from 1 to 60, from 1 to 50, from 1 to 40, from 1 to 30, from 10 to 90, from 10 to 80, from 10 to 70, from 10 to 60, from 10 to 50, from 10 to 40, from 10 to 30, from 20 to 90, preferably from 20 to 80, from 20 to 70, from 20 to 60, from 20 to 50, or from 20 to 40 mg copper/kg biomass.

In a further embodiment, isolation of Methylococcus capsulatus protein isolate from Methylococcus capsulatus biomass comprises an acid precipitation step.

In a further embodiment, the Methylococcus capsulatus protein isolate is a liquid, gel, powder, grain, flake, chunk, slurry, nugget, or crumb.

In a further embodiment, the protein isolate is produced by: disrupting Methylococcus capsulatus cells to form a cell lysate, separating and/or concentrating proteins from the cell lysate to form the Methylococcus capsulatus protein isolate, and optionally drying the protein isolate.

In a further embodiment, the Methylococcus capsulatus protein isolate is derived from Methylococcus capsulatus that is cultured and processed under good manufacturing practice (GMP) conditions.

In a further embodiment, the Methylococcus capsulatus protein isolate: (a) has gelation strength of about 10 g to about 300 g; (b) is stable as an emulsion during storage at room temperature for at least 24 hours; (c) exhibits a foam overrun of at least 200%; (d) has solubility of at least 75% at neutral pH; or any combination thereof. In further embodiments, the food composition has a gelation strength of about 10 g to about 300 g (e.g., about 15 g to about 300 g, about 25 g to about 300 g, about 50 g to about 300 g, about 15 g to about 150 g, or about 25 g to about 250 g), and the food composition is a meat-substitute, fish/seafood-substitute, extruded snack, pudding, baked good, pasta, or chip. In yet further embodiments, the food composition is stable as an emulsion during storage at room temperature for at least 24 hours, and the food composition is a sauce, dip, dressing, soup, vinaigrette, protein shake, nutritional beverage, or dairy substitute. In yet further embodiments, the food composition has a foam overrun of at least 200%, and the food composition is a meat substitute, chip, egg replacement, or baked good. In yet further embodiments, the food composition has a solubility of at least 75% (such as at least 76%, 77%, 78%, 79% or 80%) at neutral pH, and the food composition is a sauce, dip, dressing, soup, vinaigrette, dairy substitute, protein shake, nutritional beverage, protein gel, or protein supplement powder. Examples of dairy substitutes include a non-dairy milk, non-dairy creamer, non-dairy cream, non-dairy yogurt, non-dairy whipped topping, or non-dairy ice cream. Examples of meat substitutes include a patty, meatball, crumble, sausage, jerky, loaf, filet, bacon, hot dog, or nugget.

In still a further embodiment, the food composition further comprises at least one food additive, such as an emulsifying agent, viscosity increasing agent, binder, anti-caking agent, preservative, sweetener, extract, natural flavoring, flavor enhancer, fat replacement, pH control agent, leavening agent, humectant, nutrient, edible ingredient, oil, or a combination thereof.

The amount of Methylococcus capsulatus protein isolate in the food compositions disclosed herein may be in the range of about 0.1% to about 35%, such as about 0.5% to about 35% by weight.

### BRIEF DESCRIPTION OF THE DRAWINGS

FIGS. 1A-1C are flow charts showing exemplary downstream processing of biomass used in Examples 2 and 4. FIG. 1A shows a flow chart for a general process for obtaining protein isolate from bacterial biomass. FIG. 1B is a flow chart showing an exemplary microfiltration and ultrafiltration process for obtaining protein isolation from bacterial biomass. FIG. 1C is a flow chart showing an exemplary flocculent (e.g., chitosan) and ultrafiltration process for obtaining protein isolate from bacterial biomass.

FIG. 2 is a graph showing percentage crude protein of protein isolate prepared according to Example 2 from biomass of Methylococcus capsulatus Bath cultured at a low copper level (25 mg copper/kg biomass) and under a normal copper level (154 mg copper/kg biomass). B091 and B093 are biomass from a continuous fermentation at the low copper level at different time points. B089 is biomass from fermentation at the normal copper level.

FIG. 3 is a graph showing crude protein (% of dry cell weight) of biomass of Methylococcus capsulatus Bath cultured at a low copper level (38 mg copper/kg biomass), a normal copper level (154 mg copper/kg biomass), and a high copper level (371 mg/kg biomass). The numbers #24 and #28 represent two separate fermentation runs.

FIG. 4 is a graph showing crude protein, fat and ash contents of biomass from Methylococcus capsulatus Bath grown at different copper levels (i.e., 23, 80, 96 and 140 mg copper/kg biomass).

FIG. 5 shows amino acid profile analysis and protein digestibility corrected amino acid score (PDCAAS) for a control sample prepared by concentrating, heat inactivating and drying Methylococcus capsulatus biomass. (Tees002-6).

FIG. 6 shows amino acid profile analysis and PDCAAS for a sample of Methylococcus capsulatus protein isolate prepared by microfiltration/ultrafiltration method as described in Example 4 (B066).

FIG. 7 shows amino acid profile analysis and PDCAAS for a sample of Methylococcus capsulatus protein isolate prepared using chitosan and ultrafiltration method as described in Example 4 (B058).

FIG. 8 shows amino acid profile analysis and PDCAAS for a sample of Methylococcus capsulatus protein isolate prepared using chitosan/acid precipitation method as described in Example 4 (B060).

FIG. 9 shows PDCAAS scores of meat and plant-based proteins.

FIG. 10 shows gelation qualities of Methylococcus capsulatus protein isolates prepared by different methods. Left image shows that Methylococcus capsulatus protein isolate prepared by chitosan and ultrafiltration method described in Example 4 forms a weak gel. The right image shows that Methylococcus capsulatus protein isolate prepared by high speed centrifugation and acid precipitation as described in Example 4 has good gelation.

FIG. 11 shows gel strength testing using a Brookfield Texture Analyzer on Methylococcus capsulatus protein isolates produced by high speed centrifugation and acid precipitation (Acid PPT) or chitosan and ultrafiltration (chitosan/UF), whey protein concentrate (WPC), and egg white.

FIG. 12 shows PDCAAS values of a control sample and exemplary protein isolates of the present disclosure (see Example 5).

FIG. 13 shows that homogenized Methylococcus capsulatus protein isolate samples that were treated with sodium hexameta phosphate and DATEM+preheating (middle beaker) or not pre-heated (right beaker) made stable emulsions. Whey protein concentrate (left beaker) was used as a control.

FIGS. 14A-14B show two different views of protein isolate samples from left to right: whey protein concentrate, Methylococcus capsulatus protein isolate were treated with sodium hexameta phosphate and DATEM+preheating (middle), and Methylococcus capsulatus protein isolate treated with sodium hexameta phosphate and DATEM and without pre-heating show stable emulsions after 24 hours storage.

FIG. 15A-15C show foaming of Methylococcus capsulatus protein isolate solution. FIGS. 15A-15B show different perspectives of foaming of Methylococcus capsulatus protein solution following 2 minutes of whisking. FIG. 15C shows foaming of Methylococcus capsulatus protein solution produced by chitosan and ultrafiltration process (left conical tube) compared with egg white (right conical tube).

FIGS. 16A-16B show meringue made with Methylococcus capsulatus protein isolate produced by chitosan and ultrafiltration process. FIG. 16A shows the soft peaks in the meringue batter made with Methylococcus capsulatus protein isolate. FIG. 16B shows that the meringue cookies made with Methylococcus capsulatus protein isolate (right) and control meringue cookies made with egg white (left).

FIG. 17A-17B shows solubility and crude protein content (Dumas method) of Methylococcus capsulatus protein isolates made by chitosan and ultrafiltration process (UF) (without pre-heating of biomass prior to processing, heating of biomass at 65° C. for 10 seconds prior to processing, and heating of biomass at 65° C. for 5 seconds prior to processing) and high speed centrifugation and acid precipitation. Acid precipitated proteins have ˜100% solubility at pH 7.0 and 0% solubility at pH 4.3. Acid precipitated Methylococcus capsulatus protein isolate has higher protein content compared to Methylococcus capsulatus protein isolates produced by other methods.

FIG. 18 shows crude protein contents of a control sample and exemplary protein isolates of the present disclosure (see Example 5).

FIGS. 19A-19E show that Methylococcus capsulatus protein isolate or whole cell product were used in forming meat-substitute patties. Dehydrated, textured soy protein made (Dupont, Response 4310 IP Textured Soy Protein Concentrate) (FIG. 19A) was rehydrated and mixed with a Methylococcus capsulatus protein isolate to form a meat-substitute patty with fresh meat color (FIG. 19B), which darkened during cooking (FIG. 19C). The cooked meat-substitute patty made from Methylococcus capsulatus protein isolate exhibited improved texture, good gelation and water binding properties (FIG. 19D). See Example 11. Cooked meat-substitute patties made from Methylococcus capsulatus whole cell or homogenized whole cell ingredient also exhibited cooked meat color and appearance (FIG. 19E). See Example 17.

FIG. 20 shows exemplary formulations of meat-substitute hamburger patties comprising Methylococcus capsulatus protein isolate or whole cell ingredient and comparison of protein content with other plant-based protein meat-substitute patties.

FIGS. 21A-21E relate to non-dairy ice cream made with Methylococcus capsulatus protein isolate. FIG. 21A is exemplary non-dairy ice cream making process. FIG. 21B provides an exemplary strawberry non-dairy ice cream composition with control dairy strawberry ice cream composition in the right column. FIG. 21C provides an exemplary chocolate non-dairy ice cream composition with control dairy chocolate ice cream composition in the right column. FIG. 21D is an exemplary dark chocolate non-dairy ice cream composition with control dairy chocolate ice cream composition in the right column. FIG. 21E is a comparison of chocolate ice cream (top) and strawberry ice cream (bottom) made with dairy and whey protein (left) or Methylococcus capsulatus protein isolate (right). The ice cream made with Methylococcus capsulatus protein isolate had good emulsification, smooth texture, and good mouthfeel.

FIG. 22 shows crackers made with wheat flour dough (top) or wheat flour dough supplemented with Methylococcus capsulatus protein isolate (bottom) before (left) and after baking (right). The Methylococcus capsulatus protein isolate imparts a reddish color to the cracker dough, which turns brown during baking.

FIG. 23 shows a protein fortified chip made with Methylococcus capsulatus protein isolate. From top left in a clockwise direction: first image shows the flattened dough with the reddish color imparted by the Methylococcus capsulatus protein isolate; second image shows deep fried protein chips comprising Methylococcus capsulatus protein isolate; third image shows comparison of a control cooked protein fortified chip made with corn meal Masa and a protein fortified chip made with Methylococcus capsulatus protein isolate having a dark orange color after cooking. The fourth image shows baked chips fortified with Methylococcus capsulatus protein isolate (right side of image) compared with control corn chips without Methylococcus capsulatus protein isolate (left side of image).

FIG. 24 shows an exemplary process for making a dip comprising Methylococcus capsulatus protein isolate.

FIG. 25 shows an exemplary process for making a yogurt comprising Methylococcus capsulatus protein isolate or whole cell ingredient.

FIG. 26 shows an exemplary formulation of non-dairy yogurt comprising a Methylococcus capsulatus protein isolate or whole cell ingredient.

FIG. 27 is a flow chart showing exemplary downstream processing of biomass for obtaining whole cell ingredient from bacterial biomass used in Example 16.

### DETAILED DESCRIPTION

The present disclosure relates to food compositions for human consumption comprising Methylococcus capsulatus protein isolate or whole cell product. Methylococcus capsulatus is capable of converting a C1 substrate, e.g., natural gas, into food and does not compete with the human food chain. In particular, the Methylococcus capsulatus protein isolate (a) is composed of at least 70% protein; (b) has a protein digestibility corrected amino acid score (PDCAAS) of at least 0.90; and (c) has a δ¹³C of about −70‰ to about −30‰. Methylococcus capsulatus protein isolate or whole cell product is derived from Methylococcus capsulatus biomass. The use of Methylococcus capsulatus protein isolate or whole cell product offers an alternative protein source that is animal-free and may be non-genetically modified. Methylococcus capsulatus as a protein source is much more environmentally sustainable compared to plant-based and animal-based proteins, with regard to land and water use, and has scalable and consistent production. Moreover, Methylococcus capsulatus protein source offers certain health and nutritional advantages compared to meat-based and plant-based proteins, including non-allergenic, lower in saturated fatty acids, and rich in essential amino acids. In addition, Methylococcus capsulatus protein source also provides hemoprotein, thus bioavailable heme iron and enhanced flavor and/or visual appeal to the resulting food compositions. Furthermore, the δ¹³C signature of Methylococcus capsulatus protein offers traceability and verification food compositions made therefrom through the supply chain.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value and subrange within the recited range unless otherwise indicated. As used herein, the term “about” means±10% of the indicated range, value, or structure, unless otherwise indicated. The term “consisting essentially of” limits the scope of a claim to the specified materials or steps, or to those that do not materially affect the basic and novel characteristics of the claimed disclosure. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

As used herein, “C₁ substrate” or “C₁ compound” refers to an organic compound or composition containing an organic compound lacking a carbon-carbon bond. Exemplary C₁ substrates include natural gas, unconventional natural gas, biogas, methane, methanol, formaldehyde, formic acid or a salt thereof, methylated amines (e.g., methylamine, dimethylamine, trimethylamine, etc.), methylated thiols, methyl halogens (e.g., bromomethane, chloromethane, iodomethane, dichloromethane, etc.), cyanide, or any combination thereof. In certain embodiments, a C₁ substrate comprises natural gas or methane.

As used herein, the term “methanotroph,” “methanotrophic bacterium,” or “methanotrophic bacteria” refers to bacteria capable of utilizing methane or any other methane-containing C₁ substrate (e.g., natural gas), as its primary or sole carbon and energy source. Methanotrophic bacteria may be “obligate methanotrophic bacteria,” which can only utilize methane as a carbon and energy source, or “facultative methanotrophic bacteria,” which are able to use substrates other than methane as their carbon and energy source, such as methanol. Representative obligate methanotrophs include species of Methylococcus, Methylosinus, Methylomonas, Methylomicrobium, Methylobacter, (e.g., Methylococcus capsulatus Bath, Methylosinus trichosporium OB3b, Methylomonas sp. 16a, Methylomonas methanica, Methylomonas albus, Methylomicrobium alcaliphilum, Methylobacter capsulatus, Methylomonas sp. AJ-3670, Methylomicrobium buryatense 5G, Methylosinus sporium), or the like. Facultative methanotrophs include, for example, some species of Methylocella, Methylocystis, and Methylocapsa (e.g., Methylocella silvestris, Methylocella palustris, Methylocella tundrae, Methylocystis daltona SB2, Methylocystis bryophila, and Methylocapsa aurea KYG), and Methylobacterium organophilum.

As used herein, the term “Methylococcus capsulatus” refers to one or more strains of the methanotrophic bacteria Methylococcus capsulatus, including Methylococcus capsulatus (Bath), Methylococcus capsulatus (Texas), and Methylococcus capsulatus (Aberdeen). In certain embodiments, Methylococcus capsulatus refers to Methylococcus capsulatus (Bath).

As used herein, “food composition,” also known as food product or foodstuff, refers to any composition that is intended or expected to be consumed by humans as a source of nutrition and/or calories. A food composition may be a beverage, gel, or solid food. A food composition may be food product that is ready for packaging, cooking, or consumption. A food composition may be an ingredient that is combined with one or more ingredients to form a food product that is ready for packaging, cooking, or consumption. Examples of food compositions include but are not limited to meat-substitutes, egg-substitutes, baked goods, dips, butters, spreads, sauces, salad dressings, soups, dairy substitutes, puddings, pasta, chips, puffed snacks, protein shakes, nutritional beverages, protein bars, protein gels, protein supplement powders, nutritional supplement powders.

As used herein, the term “edible ingredient” refers to a food substance that is used in combination with other food substances in the preparation of food product for human consumption.

As used herein, the term “meat substitute,” also known as “meat analog,” “imitation meat,” “meat alternative,” “vegetarian meat,” “mock meat,” or “faux meat,” refers to a food composition having proteins partially derived from animal-based proteins but supplemented with non-animal protein source(s) or having proteins wholly derived from non-animal source(s). In certain embodiments, the meat substitute has proteins wholly derived from non-animal source(s). Meat substitutes may be in various forms, including a patty, meatball, crumble, sausage, jerky, loaf, filet, bacon, hot dog, or nugget.

As used herein, the term “baked goods” refers to a food composition that is prepared by baking in an oven and usually contains a leavening agent. Baked goods include, but are not limited to cookies, brownies, cakes, meringue, pies, pastries, buns, breads, and quick breads (e.g., crackers).

As used herein, the term “dairy substitute,” “dairy alternative,” “non-dairy product” or “dairy free product” refers to a food composition that contains 0.5% or less milk by weight, in the form of casein/caseinates (milk protein). Dairy substitutes include and are not limited to non-dairy milk, non-dairy creamer, non-dairy cream, non-dairy yogurt, non-dairy whipped topping, and non-dairy ice cream.

As used herein, the term “nutritional supplement” refers to a composition intended to supplement the diet by providing specific nutrients as opposed to bulk calories. A nutritional supplement may contain any one or more of vitamins, minerals, fiber, fatty acids and amino acids. A nutritional supplement may be in the form of a tablet, powder, gel, or liquid.

As used herein, the term “good manufacturing practice” or “GMP” refers to regulations promulgated by the US Food and Drug Administration under the authority of the Federal Food, Drug, and Cosmetic Act in 21 CFR 110 (for human food) and 111 (for dietary supplements) or comparable regulations set forth in jurisdictions outside the U.S that describe conditions and practices that are necessary for the manufacturing, processing, packing or storage of food to ensure its safety and wholesomeness.

### Fermentation

The present disclosure provides food compositions for human consumption comprising Methylococcus capsulatus protein isolate derived from M. capsulatus biomass. M. capsulatus may be grown by batch culture or continuous culture methodologies. In certain embodiments, the cultures are grown in a controlled culture unit, such as a fermentor, bioreactor, hollow fiber cell, or the like. Generally, cells in log phase are often responsible for the bulk production of a product or intermediate of interest in some systems, whereas stationary or post-exponential phase production can be obtained in other systems.

A classical batch culturing method is a closed system in which the media composition is set when the culture is started and is not altered during the culture process. That is, media is inoculated at the beginning of the culturing process with one or more microorganisms of choice and then are allowed to grow without adding anything to the system. As used herein, a “batch” culture is in reference to not changing the amount of a particular carbon source initially added, whereas control of factors such as pH and oxygen concentration can be monitored and altered during the culture. In batch systems, metabolite and biomass compositions of the system change constantly up to the time the culture is terminated. Within batch cultures, bacterial cells will generally move from a static lag phase to a high growth logarithmic phase to a stationary phase where growth rate is reduced or stopped (and will eventually lead to cell death if conditions do change).

A fed-batch system is a variation on the standard batch system in which a carbon substrate of interest is added in increments as the culture progresses. Fed-batch systems are useful when cell metabolism is likely to be inhibited by catabolite repression and when it is desirable to have limited amounts of substrate in the media. Since it is difficult to measure actual substrate concentration in fed-batch systems, an estimate is made based on changes of measureable factors such as pH, dissolved oxygen, and the partial pressure of waste gases. Batch and fed-batch culturing methods are common and known in the art (see, e.g., Thomas D. Brock, Biotechnology: A Textbook of Industrial Microbiology, 2^{nd} Ed. (1989) Sinauer Associates, Inc., Sunderland, MA; Deshpande, Appl. Biochem. Biotechnol. 36:227, 1992).

Continuous cultures are “open” systems in the sense that defined culture media is continuously added to a bioreactor while an equal amount of used (“conditioned”) media is removed simultaneously for processing. Continuous cultures generally maintain the cells at a constant high, liquid phase density where cells are primarily in logarithmic growth phase. Alternatively, continuous culture may be practiced with immobilized cells (e.g., biofilm) where carbon and nutrients are continuously added and valuable products, by-products, and waste products are continuously removed from the cell mass. Cell immobilization may be achieved with a wide range of solid supports composed of natural materials, synthetic materials, or a combination thereof.

Continuous or semi-continuous culture allows for the modulation of one or more factors that affect cell growth or end product concentration. For example, one method may maintain a limited nutrient at a fixed rate (e.g., carbon source, nitrogen) and allow all other parameters to change over time. In other embodiments, several factors affecting growth may be continuously altered while cell concentration, as measured by media turbidity, is kept constant. The goal of a continuous culture system is to maintain steady state growth conditions while balancing cell loss due to media being drawn off against the cell growth rate. Methods of modulating nutrients and growth factors for continuous culture processes and techniques for maximizing the rate of product formation are well known in the art (see, e.g., Thomas D. Brock, Biotechnology: A Textbook of Industrial Microbiology, 2^{nd} Ed. (1989) Sinauer Associates, Inc., Sunderland, MA; Deshpande, Appl. Biochem. Biotechnol. 36:227 1992).

In certain embodiments, culture medium includes a carbon substrate as a source of energy for a methanotrophic bacterium. Suitable substrates include C₁ substrates, such as methane, methanol, formaldehyde, formic acid (formate), carbon monoxide, carbon dioxide, methylated amines (methylamine, dimethylamine, trimethylamine, etc.), methylated thiols, or methyl halogens (bromomethane, chloromethane, iodomethane, dichloromethane, etc.). In certain embodiments, culture media may comprise a single C₁ substrate as the sole carbon source for a methanotrophic bacterium, or may comprise a mixture of two or more C₁ substrates (mixed C₁ substrate composition) as multiple carbon sources for a methanotrophic bacterium. In certain embodiment, natural gas (which primarily contains methane) or unconventional natural gas may be used as a carbon source.

During bacterial culture, the pH of the fermentation mixtures will generally be regulated to be between about 6 and about 8, such as between about 6 and about 7, between about 7 and about 8, or between about 6.5 and 7.5.

During bacterial culture, the temperature is maintained to be in the range optimal for the cultured bacterium. For example, for M. capsulatus Bath, the temperature may be between 40° C. and 45° C., such as 42° C.

Preferably, M. capsulatus may be cultured using methane as its carbon source, air or pure oxygen for oxygenation, and ammonia as the nitrogen source. In certain embodiments, a carbon feedstock comprising methane used for culturing M. capsulatus is natural gas or unconventional natural gas. In addition to these substrates, the bacterial culture will typically require water, phosphate, and several minerals such as magnesium, calcium, potassium, irons, copper, zinc, manganese, nickel, cobalt and molybdenum. Exemplary culture media include Higgins minimal nitrate salts medium (NSM) or MM-W1 medium, master mix feed (MMF) as described in Example 1, MMF1.1, medium MMS1.0, or AMS medium. The copper concentrations of these media may be adjusted according to low copper conditions as described above.

The composition of medium MMS 1.0 is as follows: 0.8 mM MgSO₄·7H₂O, 30 mM NaNO₃, 0.14 mM CaCl₂), 1.2 mM NaHCO₃, 2.35 mM KH₂PO₄, 3.4 mM K₂HPO₄, 20.7 μM Na₂MoO₄·2H₂O, 6 μM CuSO₄·5H₂O, 10 μM Fe^{III}—Na-EDTA, and 1 mL per liter of a trace metals solution (containing per liter: 500 mg FeSO₄·7H₂O, 400 mg ZnSO₄·7H₂O, 20 mg MnCl₂·7H₂O, 50 mg CoCl₂·6H₂O, 10 mg NiCl₂·6H₂O, 15 mg H₃BO₃, 250 mg EDTA). The final pH of the media is 7.0±0.1.

The AMS medium contains the following per liter: 10 mg NH₃, 75 mg H₃PO₄·2H₂O, 380 mg MgSO₄·7H₂O, 100 mg CaCl₂·2H₂O, 200 mg K₂SO₄, 75 mg FeSO₄·7H₂O, 1.0 mg CuSO₄·5H₂O, 0.96 mg ZnSO₄·7H₂O, 120 μg CoCl₂·6H₂O, 48 μg MnCl₂·4H₂O, 36 μg H₃BO₃, 24 μg NiCl₂·6H₂O and 1.20 μg NaMoO₄·2H₂O.

The composition of medium MMF1.1 is as follows: 0.8 mM MgSO₄·7H₂O, 40 mM NaNO₃, 0.14 mM CaCl₂), 6 mM NaHCO₃, 4.7 mM KH₂PO₄, 6.8 mM K₂HPO₄, 20.7 μM Na2MoO₄·2H₂O, 6 μM CuSO₄·5H₂O, 10 μM Fe^{III}—Na-EDTA, and 1 mL per liter of trace metals solution (containing, per liter 500 mg FeSO₄·7H₂O, 400 mg ZnSO₄·7H₂O, 20 mg MnCl₂·7H₂O, 50 mg COCl₂·6H₂O, 10 mg NiCl₂·6H₂O, 15 mg H₃BO₃, 250 mg EDTA).

Suitable fermenters for culturing methanotrophic bacteria may be of the loop-type or air-lift reactors. Exemplary fermenters include U-loop fermenters (see U.S. Pat. No. 7,579,163, WO2017/218978), serpentine fermenters (see WO 2018/132379), and Kylindros fermenters (see WO 2019/0366372).

In certain embodiments, M. capsulatus is cultured under good manufacturing practice (GMP) conditions.

In certain embodiments, M. capsulatus is cultured as an isolated culture without the presence of another organism. In certain other embodiments, M. capsulatus may be grown with one or more heterologous organisms that may aid with growth of the M. capsulatus bacterium. For example, Methylococcus capsulatus Bath may be cultured with Cupriavidus sp., Aneurinibacillus danicus, or both, and optionally in combination with Brevibacillus agri.

In certain embodiments, M. capsulatus may be cultured (especially continuously cultured) under low copper conditions. The present inventors discovered that methanotrophic bacteria cultured under such conditions produced biomass with higher crude protein, lower lipid, and/or lower ash contents than the bacteria cultured under normal or high copper conditions (see Example 2). Such biomass allows the production of protein isolate having high crude protein, increased yield, and/or minimal nucleic acid. Without wishing to be bound to any theory, the present inventors hypothesize that the increase in crude protein of biomass from methanotrophic bacteria cultured under low copper conditions may be due to the reduction in the amount of the internal membrane structure (including lipid and membrane proteins) induced by low copper conditions.

The term “low copper conditions” refers to continuous culture conditions where the amount (or level) of copper in a continuous culture is at most 100 mg copper (i.e., elemental copper or copper element) per kg dry cell weight (DCW).

“Continuous culture conditions” refers to conditions under which M. capsulatus is cultured in a continuous culturing system wherein a defined culture medium (or its component(s)) is continuously added to the system while an equal amount of used medium is removed simultaneously for processing.

“Continuous culture” refers to the mixture of a culture medium and bacteria cultured in the medium under continuous culture conditions.

“Dry cell weight (DCW)” refers to the dry weight of biomass harvested from a bacterial culture.

A specified amount of copper element is typically provided by a corresponding or equivalent amount of a copper salt that contains the same number of mole of copper element. For example, 100 mg copper is about 1.57 mmol, and may be provided by about 394 mg CuSO₄·5H₂O.

The term “high copper conditions” refers to continuous culture conditions where the amount of copper in a continuous culture is more than 200 mg copper per kg dry cell weight (DCW).

Specified copper conditions are typically set up by controlling Cu feed rates in view of DCW harvest rates. For example, for a low copper (Cu) concentration of 50 μg Cu/g of DCW (dry cell weight) and harvest rate 5 g/L/h of DCW, Cu (such as provided by CuSO₄·5H₂O) feed should be 250 μg Cu/L/h.

In certain embodiments, copper concentrations may be controlled by the use of a device (e.g., a pump) to feed a continuous culture at a defined rate.

In certain embodiments, the copper level under low copper conditions is from 1 to 100, from 1 to 10, from 10 to 20, from 20 to 30, from 30 to 40, from 40 to 50, from 50 to 60, from 60 to 70, from 70 to 80, from 80 to 90, from 90 to 100, from 1 to 90, from 1 to 80, from 1 to 70, from 1 to 60, from 1 to 50, from 1 to 40, from 1 to 30, from 10 to 90, from 10 to 80, from 10 to 70, from 10 to 60, from 10 to 50, from 10 to 40, from 10 to 30, from 20 to 90, preferably from 20 to 80, from 20 to 70, from 20 to 60, from 20 to 50, or from 20 to 40 mg copper/kg biomass. In certain other embodiments, M. capsulatus may be cultured under normal copper conditions. The term “normal copper conditions” refers to continuous culture conditions where the amount of copper in a continuous culture is in the range of 100 mg to 200 mg copper per kg dry cell weight (DCW). In certain embodiments, the copper level under normal copper conditions is from 100 to 180, from 100 to 170, from 100 to 160, from 100 to 150, from 100 to 140, from 100 to 130 mg copper/kg biomass.

In certain other embodiments, M. capsulatus may be cultured under high copper conditions. The term “high copper conditions” refers to continuous culture conditions where the amount of copper in a continuous culture is more than 200 mg copper per kg dry cell weight (DCW). In certain embodiments, the copper level under high copper conditions is from 200 to 800, from 200 to 700, from 200 to 600, from 200 to 500, or from 200 to 400 mg copper/kg biomass.

Exemplary culturing conditions of M. capsulatus are provided in Examples 1 and 3. Additional description about culturing methanotrophic biomass under low copper conditions may be found in the U.S. provisional application titled “Methods For Culturing Methanotrophic Bacteria and Isolating Proteins From Bacterial Biomass” filed on Oct. 7, 2019.

### Biomass, Protein Isolate, and Whole Cell Product

M. capsulatus protein isolate used in food compositions provided is generated by purifying proteins from biomass obtained as disclosed herein.

“Biomass” or “bacterial biomass” refers to organic material collected from bacterial culture. Biomass primarily (i.e., more than 50% w/w) comprises bacterial cells, but may include other materials such as lysed bacterial cells, bacterial cell membranes, inclusion bodies, and extracellular material (e.g., products secreted or excreted into the culture medium), or any combination thereof that are collected (e.g., via centrifugation) from bacterial fermentation along with bacterial cells. Preferably, the biomass includes more than 60%, 70%, 75%, 80%, 85%, 90% or 95% cells collected from bacterial fermentation.

Biomass may be collected or harvested from bacterial culture by various techniques, such as sedimentation, microfiltration, ultrafiltration, and spray drying. Preferably, biomass is harvested from bacterial culture by centrifugation (e.g., at 4,000×g for 10 minutes at 10° C.). For example, a fermentation broth (cells and liquid) may be collected and centrifuged. After centrifugation, the liquid can be discarded, and the precipitated cells may be saved and optionally lyophilized.

In certain embodiments, bacterial biomass consists essentially of or consists of the biomass harvested from M. capsulatus cultured under low copper conditions has a copper level no more than 100 mg copper per kg DCW (mg/kg). In certain embodiments, the biomass of M. capsulatus has a copper level from 1 to 100, from 1 to 10, from 10 to 20, from 20 to 30, from 30 to 40, from 40 to 50, from 50 to 60, from 60 to 70, from 70 to 80, from 80 to 90, from 90 to 100, from 1 to 90, from 1 to 80, from 1 to 70, from 1 to 60, from 1 to 50, from 1 to 40, from 1 to 30, from 10 to 90, from 10 to 80, from 10 to 70, from 10 to 60, from 10 to 50, from 10 to 40, from 10 to 30, from 20 to 90, from 20 to 80, from 20 to 70, from 20 to 60, from 20 to 50, or from 20 to 40 mg copper/kg DCW.

In certain embodiments, the biomass of M. capsulatus cultured under normal or low copper conditions has at least 60% crude protein, such as at least 61%, 62%, 63%, 64%, 65%, 66%, 67%, 68%, 69%, 70%, 71%, 72%, 73%, 74%, 75%, 76%, 77%, 78%, 79%, 80%, or 81% crude protein. “Crude protein,” “crude protein content,” “crude protein concentration,” or “percentage crude protein” is a measurement of nitrogen in a protein sample. The amount of nitrogen is indicative of the amount of protein in the sample. The crude protein content of biomass or protein isolate disclosed herein is measured by the Dumas method. In certain embodiments, the biomass of M. capsulatus is composed of about 60% to about 99%, about 65% to about 99%, about 71% to about 99%, about 75% to about 99%, about 80% to about 99%, 82% to about 99%, about 60% to about 95%, about 65% to about 95%, about 71% to about 95%, about 75% to about 95%, about 80% to about 95%, about 82% to about 95%, about 60% to about 90%, about 65% to about 90%, about 71% to about 90%, about 75% to about 90%, about 80% to about 90%, about 82% to about 90%, about 60% to about 85%, about 65% to about 85%, about 71% to about 85%, about 75% to about 85%, about 60% to about 80%, about 65% to about 80%, or about 71% to about 80%, crude protein.

In certain embodiments, the biomass of M. capsulatus cultured under normal or low copper conditions has at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% true protein. “True protein,” “true protein content,” “true protein concentration” or “percentage true protein” is a measurement of crude protein minus the non-protein nitrogen content in a protein sample. In certain embodiments, the biomass of M. capsulatus is composed of about 50% to about 99%, about 55% to about 99%, about 60% to about 99%, about 65% to about 99%, about 70% to about 99%, about 75% to about 99%, about 80% to about 99%, about 50% to about 95%, about 55% to about 95%, about 60% to about 95%, about 65% to about 95%, about 70% to about 95%, about 75% to about 95%, about 80% to about 95%, about 50% to about 90%, about 55% to about 90%, about 60% to about 90%, about 65% to about 90%, about 70% to about 90%, about 75% to about 90%, about 80% to about 90%, about 50% to about 85%, about 55% to about 85%, about 60% to about 85%, about 65% to about 85%, about 70% to about 85%, about 75% to about 85%, about 50% to about 80%, about 55% to about 80%, about 60% to about 80%, about 65% to about 80%, about 70% to about 80%, about 50% to about 75%, about 55% to about 75%, about 60% to about 75%, about 50% to about 70%, about 55% to about 70%, about 60% to about 70%, about 50% to about 65%, about 55% to about 65%, or about 50% to about 60%, true protein.

In certain embodiments, the biomass of M. capsulatus cultured under low copper conditions has at most 14% such as at most 13%, 12%, or 11% ash. “Ash” is material left over in a sample that is burned (e.g., in furnace for 12-18 hours or overnight at 550° C.).

In certain embodiments, the biomass of M. capsulatus cultured under normal or low copper conditions has at most 10%, such as at most 9%, 8%, 7%, 6%, or 5% nucleic acids. The nucleic acid content of biomass or protein isolate disclosed herein is measured using a Lucigen Masterpure Complete DNA & RNA Purification Kit MC85200.

In certain embodiments, the biomass of M. capsulatus cultured under normal or low copper conditions has at most 15%, 14%, 13%, 12%, 11%, 10%, 9%, 8%, 7.5%, 7%, 6%, or 5% crude fat. Crude fat may be measured by acid hydrolysis followed by organic solvent extraction. Briefly, fats or lipids in the bacterial biomass and/or the biomass of methanotrophic bacterium are first broken down via acid hydrolysis before being extracted via a solvent (e.g., ether or hexane). The solvent is then evaporated, and the material that remains is referred to “crude fat.”

In certain embodiments, the biomass of M. capsulatus cultured under normal or low copper conditions has at least 0.01 mg heme/g protein, such as at least 0.05 mg heme/g protein. The amount of heme in M. capsulatus biomass or protein isolate as disclosed herein is measured by a method based on the conversion of heme to the fluorescent porphyrin derivative via removal of the heme iron under acidic conditions (Sassa, Journal of Experimental medicine 143: 305-315, 1976). In some embodiments, the biomass of M. capsulatus has at least 0.01 mg, at least 0.5 mg, or at least 1.0 mg, heme per gram protein. In certain embodiments, the biomass of M. capsulatus contains 0.01 to 10 mg heme/g protein, such as 0.1 to 1, 1 to 5, 5 to 10, 0.2 to 0.5, 0.5 to 1, 1 to 2, or 2 to 4, mg heme/g protein.

Heme is a coordination complex of an iron ion coordinated to a porphyrin molecule. M. capsulatus produces hemoproteins, which contain at least one heme that is tightly bound in stoichiometric amount to the proteins (e.g., with a binding constant in the range of 10⁻⁸ to 10⁻¹⁵ M) and can often be identified by their red color. Hemoproteins may be measured by measuring a peak at 410-415 nm and a peak at 500-550 nm via UV-visible absorption spectroscopy. Hemoproteins produced by M. capsulatus include multi-heme c-type cytochromes and di-heme cytochrome c peroxidase (see e.g., Karlsen et al., FEMS Microbiol Lett 323: 97-104, 2011).

The iron ion coordinated to a porphyrin molecule in heme is referred to as “heme iron” or “heme-iron.” Heme-iron as a dietary source of iron is more easily absorbed than non-heme iron and in a pathway that is distinct from that of non-heme-iron. Heme-iron remains soluble in the high pH environment of the upper small bowel, in contrast to inorganic, non-heme iron. The amount of heme iron can be calculated based on the 1:1 molar ratio between heme and heme iron after the amount of heme is measured.

In certain embodiments where M. capsulatus is cultured with one or more heterologous organisms, such as Cupriavidus sp., Aneurinibacillus danicus or both and optionally in combination with Brevibacillus agri, the bacterial biomass may comprise biomass from the heterologous organism(s) in addition to biomass from M. capsulatus.

Preferably, the bacterial biomass comprises primarily (i.e., more than 50%, such as more than 55%, more than 60%, more than 65%, more than 70%, more than 75%, more than 80%, more than 85% or more than 90% by weight) biomass from M. capsulatus.

In certain embodiments where M. capsulatus is cultured with one or more heterologous organisms, the bacterial biomass and/or the biomass of M. capsulatus has a copper level no more than 100 mg copper per kg DCW (mg/kg). In certain embodiments, the bacterial biomass and/or the biomass of M. capsulatus has a copper level from 1 to 100, from 1 to 10, from 10 to 20, from 20 to 30, from 30 to 40, from 40 to 50, from 50 to 60, from 60 to 70, from 70 to 80, from 80 to 90, from 90 to 100, from 1 to 90, from 1 to 80, from 1 to 70, from 1 to 60, from 1 to 50, from 1 to 40, from 1 to 30, from 10 to 90, from 10 to 80, from 10 to 70, from 10 to 60, from 10 to 50, from 10 to 40, from 10 to 30, from 20 to 90, from 20 to 80, from 20 to 70, from 20 to 60, from 20 to 50, or from 20 to 40 mg copper/kg DCW.

In certain embodiments where M. capsulatus is cultured with one or more heterologous organisms, the bacterial biomass and/or the biomass of M. capsulatus cultured under normal or low copper conditions has at least 60% crude protein, such as at least 61%, 62%, 63%, 64%, 65%, 66%, 67%, 68%, 69%, 70%, 71%, 72%, 73%, 74%, 75%, 76%, 77%, 78%, 79%, 80%, or 81% crude protein, such as about 60% to about 99%, about 65% to about 99%, about 71% to about 99%, about 75% to about 99%, about 80% to about 99%, 82% to about 99%, about 60% to about 95%, about 65% to about 95%, about 71% to about 95%, about 75% to about 95%, about 80% to about 95%, about 82% to about 95%, about 60% to about 90%, about 65% to about 90%, about 71% to about 90%, about 75% to about 90%, about 80% to about 90%, about 82% to about 90%, about 60% to about 85%, about 65% to about 85%, about 71% to about 85%, about 75% to about 85%, about 60% to about 80%, about 65% to about 80%, or about 71% to about 80%, crude protein.

In certain embodiments where M. capsulatus is cultured with one or more heterologous organisms, the bacterial biomass and/or the biomass of M. capsulatus cultured under normal or low copper conditions has at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% true protein, such as about 50% to about 99%, about 55% to about 99%, about 60% to about 99%, about 65% to about 99%, about 70% to about 99%, about 75% to about 99%, about 80% to about 99%, about 50% to about 95%, about 55% to about 95%, about 60% to about 95%, about 65% to about 95%, about 70% to about 95%, about 75% to about 95%, about 80% to about 95%, about 50% to about 90%, about 55% to about 90%, about 60% to about 90%, about 65% to about 90%, about 70% to about 90%, about 75% to about 90%, about 80% to about 90%, about 50% to about 85%, about 55% to about 85%, about 60% to about 85%, about 65% to about 85%, about 70% to about 85%, about 75% to about 85%, about 50% to about 80%, about 55% to about 80%, about 60% to about 80%, about 65% to about 80%, about 70% to about 80%, about 50% to about 75%, about 55% to about 75%, about 60% to about 75%, about 50% to about 70%, about 55% to about 70%, about 60% to about 70%, about 50% to about 65%, about 55% to about 65%, or about 50% to about 60%, true protein.

In certain embodiments where M. capsulatus is cultured with one or more heterologous organisms, the bacterial biomass and/or the biomass of M. capsulatus bacterium cultured under normal or low copper conditions has at most 14% such as at most 13%, 12%, or 11% ash.

In certain embodiments where M. capsulatus is cultured with one or more heterologous organisms, the bacterial biomass and/or the biomass of M. capsulatus bacterium cultured under normal or low copper conditions has at most 10%, such as at most 9%, 8%, 7%, 7.5%, 6%, or 5% nucleic acid.

In certain embodiments where M. capsulatus is cultured with one or more heterologous organisms, the bacterial biomass and/or the biomass of M. capsulatus cultured under normal or low copper conditions has at most 15%, 14%, 13%, 12%, 11%, 10%, 9%, 8%, 7%, 6%, or 5% crude fat.

In certain embodiments where M. capsulatus is cultured with one or more heterologous organisms, the bacterial biomass and/or the biomass of M. capsulatus cultured under normal or low copper conditions has at least 0.01 mg heme/g protein.

In some embodiments, the biomass of M. capsulatus has at least 0.01 mg, at least 0.05 mg, at least 0.1 mg, at least 0.5 mg, at least 1.0 mg heme per gram protein in the biomass. In certain embodiments, the biomass of M. capsulatus contains 0.01 to 10 mg heme/g protein, such as 0.05 to 0.1, 0.1 to 1, 1 to 5, 5 to 10, 0.2 to 0.5, 0.5 to 1, 1 to 2, 2 to 4, or 0.05 to 5 mg heme/g protein.

In certain embodiments, the biomass is harvested from M. capsulatus cultured under GMP conditions.

The term “protein isolate” refers to a composition that comprises primarily proteins isolated, extracted or purified from a bacterial biomass that comprises primarily, consisting essentially of, or consisting of a biomass of M. capsulatus. A composition that comprises primarily proteins isolated from a bacterial biomass refers to a composition of which more than 50% (e.g., more than 55%, 60%, 70%, 75% or 80%) by weight is proteins from the bacterial biomass. Through the use of protein isolation, extraction or purification technique(s) (e.g., microfiltration, ultrafiltration, chitosan flocculation, centrifugation, acid precipitation, and combinations thereof), protein isolate has higher protein content (e.g., measured by percentage crude protein) than the bacterial biomass from which the protein isolate is prepared. However, protein isolation, extraction or purification does not need to be to the extent that individual proteins are separated from each other. Instead, protein isolate in general comprises a mixture of proteins isolated, extracted or purified from a bacterial biomass in which at least some of the other components, especially at least some, preferably most (e.g., more than 50%, 60%, 70%, 80%, 90% or 95%) or all, of solid components (e.g., cell debris and/or cell wall), in the bacterial biomass are removed.

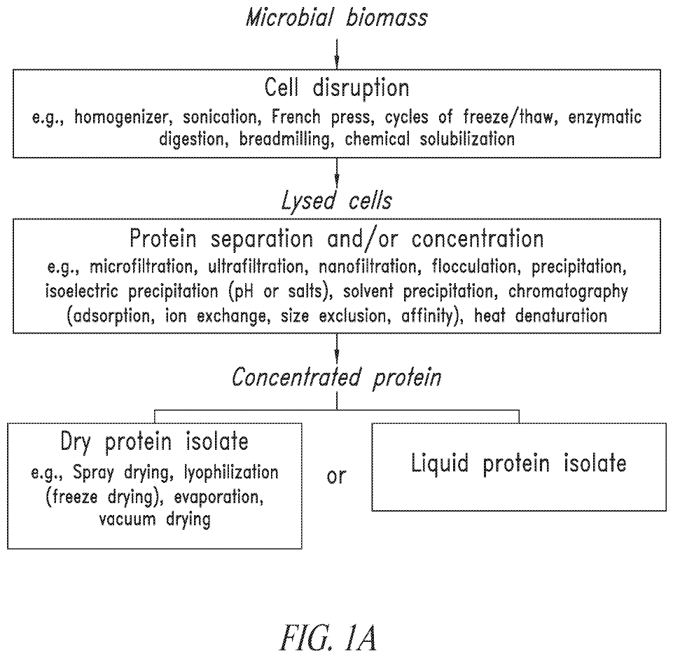

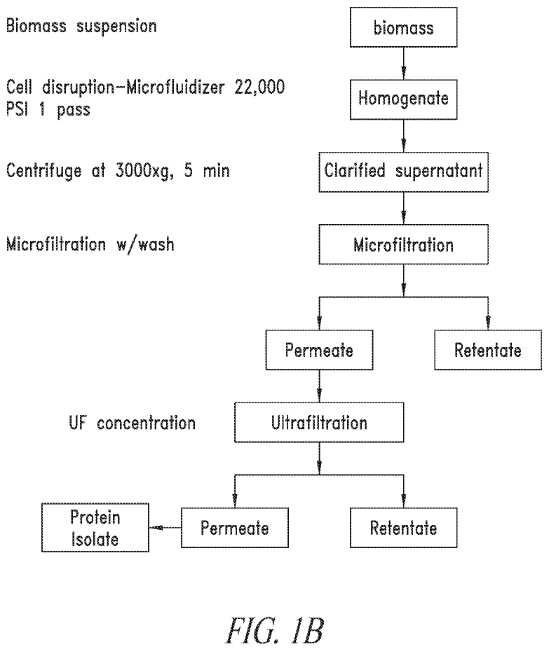

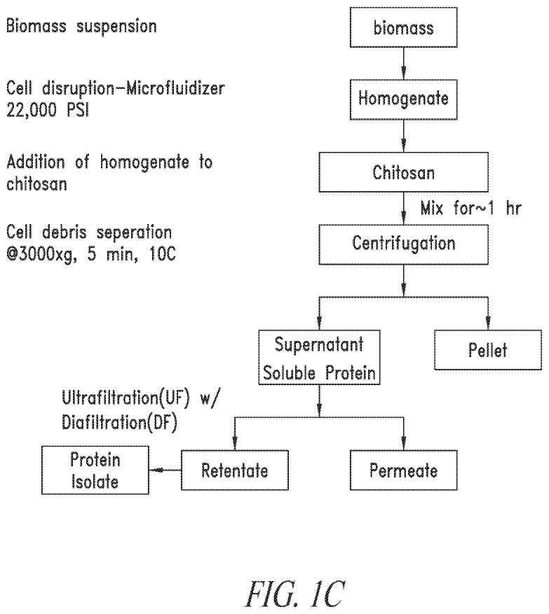

In general, biomass harvested from a culture of a methanotrophic bacterium goes through a cell disruption step (e.g., homogenization, beadmilling, freeze/thaw cycles, enzymatic digestion, sonication, French press, and chemical solubilization) to generate a lysate first followed by protein separation and/or concentration step(s) (e.g., flocculation, microfiltration, ultrafiltration, nanofiltration, precipitation, isoelectric precipitation (via pH or salts), solvent precipitation, chromatographic methods based on adsorption, ion exchange chromatography, size exclusion chromatography, or affinity, and heat denaturation) of the lysate to generate protein isolate. The resulting protein isolate may be a liquid protein isolate or further dried (e.g., via spray drying, lyophilization, evaporation, vacuum drying) to obtain dry protein isolate. A schematic representation is shown in FIG. 1A.

An exemplary workflow of preparing protein isolate is to homogenize bacterial biomass (e.g., via a microfluidizer), centrifuge the lysate (also referred to as homogenate) to obtain clarified supernatant, subject the supernatant to microfiltration, subject the resulting permeate to ultrafiltration, and lyophilize the resulting retentate to obtain protein isolate as a dry powder. A schematic representation of a specific example of the workflow used in Examples 2 and 4 is shown in FIG. 1B.

Another exemplary workflow of preparing protein isolate is to homogenize bacterial biomass (e.g., via a microfluidizer), add a flocculant (e.g., chitosan) to the homogenate, centrifuge the resulting mixture to remove cell debris and obtain clarified supernatant, subject the clarified supernatant to ultrafiltration, and lyophilize the resulting retentate to obtain protein isolate as a dry powder. A schematic representation of a specific example of the workflow used in Example 4 is shown in FIG. 1C.

A further exemplary workflow of preparing protein isolate is to homogenize bacterial biomass (e.g., via a microfluidizer), add a flocculant to the homogenate, centrifuge the resulting mixture to remove nucleic acids and/or cell debris and obtain clarified supernatant, subject the clarified supernatant to acid precipitation (e.g., adjusting pH with H₂SO₄ to about 4.5), optionally wash precipitated proteins, neutralize and re-suspend precipitated proteins (e.g., adjust pH with sodium hydroxide to 7), and lyophilize re-suspended proteins to obtain protein isolate as a dry powder. A specific example of this workflow is described in Example 4.

Another exemplary workflow of preparing protein isolate is to homogenize bacterial biomass (e.g., via a microfluidizer), centrifuge the homogenate to remove cell debris and obtain clarified supernatant, subject the clarified supernatant to acid precipitation (e.g., adjusting pH with H₂SO₄ to about 4.5), optionally wash precipitated proteins, neutralize and re-suspend precipitated proteins (e.g., adjust pH with sodium hydroxide to 7), and lyophilize re-suspended proteins to obtain protein isolate as a dry powder. A specific example of this workflow is described in Example 4.

Flocculants, especially cationic flocculants, that may be used in preparing protein isolate to reduce nucleic acid and/or cell debris include chitosan (e.g., chitosan derived from shellfish or fungal source), poly-L-lysine, polyethylenimine (PEI), DEAE (diethylaminoethyl ion exchange resin), DEAE-dextran hydrochloride, amidated pectin (e.g., amidated low methoxyl pectin), Tramfloc 860 series (alkylamine epichlorohydrin), pDADMACs (diallydimethylammonium chloride), lsinglass, gelatin, and egg white. Preferably, a flocculant is chitosan, poly-L-lysine, DEAE, alkylamine epichlorhydrins, pDADMACs, or any combination thereof.

As disclosed above, culturing methanotrophic bacteria under low copper conditions not only increases the crude protein content in bacterial biomass, it may also increase the yield of protein isolate. Accordingly, the yield of protein isolate prepared from biomass of a methanotrophic bacterium cultured under low copper conditions may be higher than that the yield of protein isolate prepared from biomass of the methanotrophic bacterium cultured under normal or high copper conditions. In certain embodiments, the ratio of the yield of protein isolate prepared from biomass of a methanotrophic bacterium cultured under low copper conditions to the yield of protein isolate prepared from biomass of the methanotrophic bacterium cultured under normal or high copper conditions (e.g., at the copper level of 150 mg copper per kg DCW) is at least 1.2, 1.3, 1.4, 1.5, 1.6, 1.7, 1.8, 1.9, or preferably at least 2.0, 2.1, 2.2, 2.3, 2.4, or 2.5.

The “yield of protein isolate” refers to the percentage of protein from biomass homogenate retained in protein isolate. In other words, the yield of protein isolate is the percentage of protein in protein isolate when setting the percentage of protein in the biomass homogenate from which the protein isolate is prepared to be 100%. Biomass homogenate is a mixture resulting from homogenizing biomass. The protein content may be measured by the BCA method (Smith et al., Anal Biochem. 150(1):76-85, 1985), such as using ThermoFisher Scientific Pierce BCA Protein Assay Kit).

In certain embodiments, the yield of the protein isolate is at least 10%, such as at least 11%, 12%, 13%, 14%, 15%, 16%, 17%, 18%, 19%, preferably at least 20%, 21%, 22%, 23%, 24% or 25%.

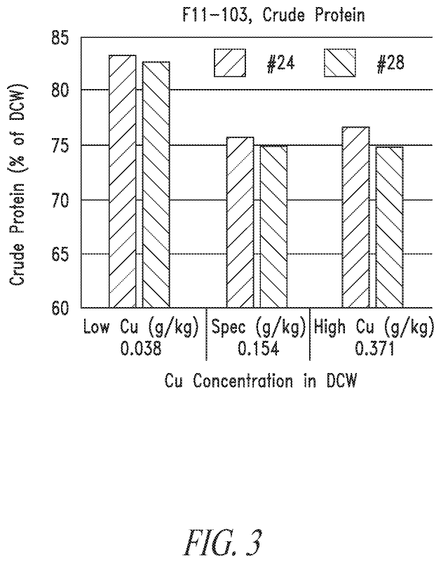

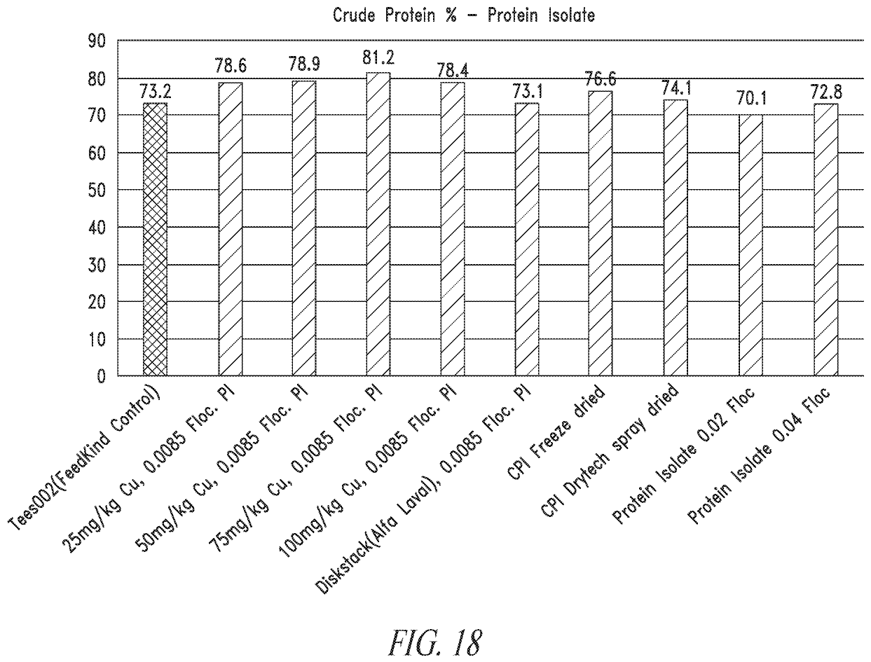

In certain embodiments, Methylococcus capsulatus protein isolate used in food compositions of the present disclosure is composed of at least 70% crude protein. In certain embodiments, the Methylococcus capsulatus protein isolate is composed of at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight crude protein. In certain embodiments, the Methylococcus capsulatus protein isolate is composed of about 70% to about 99%, about 71% to about 99%, about 75% to about 99%, about 80% to about 99%, about 81% to about 99%, about 85% to about 99%, about 90% to about 99%, about 70% to about 95%, about 71% to about 95%, about 75% to about 95%, about 80% to about 95%, about 70% to about 90%, about 71% to about 90%, about 75% to about 90%, about 80% to about 90%, about 70% to about 80% weight protein, or about 71% to about 80% by weight crude protein. As shown in FIGS. 3 and 18, Methylococcus capsulatus protein isolate prepared in different ways show crude protein content of more than 70% by weight.

In certain embodiments, Methylococcus capsulatus protein isolate used in food compositions of the present disclosure is composed of at least 60% true protein. In certain embodiments, Methylococcus capsulatus protein isolate is composed of at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight true protein. In certain embodiments, the Methylococcus capsulatus protein isolate is composed of about 60% to about 99%, about 60% to about 95%, about 60% to about 90%, about 60% to about 85%, about 60% to about 80%, about 60% to about 75%, about 60% to about 70%, about 65% to about 99%, about 65% to about 95%, about 65% to about 90%, about 65% to about 85%, about 65% to about 80%, about 65% to about 75%, about 70% to about 99%, about 70% to about 95%, about 70% to about 90%, about 70% to about 85%, about 70% to about 80% by weight, about 75% to about 99%, about 75% to about 95%, about 75% to about 90%, about 75% to about 85%, about 80% to about 99%, about 80% to about 95%, or about 80% to about 90% true protein.

In certain embodiments, Methylococcus capsulatus protein isolate has less than about 10%, 9%, 8%, 7%, 6%, 5%, 4%, 3%, 2%, or 1% nucleic acids. The nucleic acid content of biomass or protein isolate disclosed herein is measured using a Lucigen Masterpure Complete DNA & RNA Purification Kit MC85200.

In certain embodiments, Methylococcus capsulatus protein isolate has less than about 10%, 9%, 8%, 7%, 6%, 5%, 4%, 3%, 2%, or 1% by weight ash. “Ash” is material left over in a sample that is burned (e.g., in furnace for 12-18 hours or overnight at 550° C.).

In certain embodiments, Methylococcus capsulatus protein isolate has less than about 15%, 14%, 13%, 12%, 11%, 10%, 9%, 8%, 7%, 6%, 5%, 4%, 3%, 2%, or 1% by weight crude fat.

In certain embodiments, Methylococcus capsulatus protein isolate has at least 0.01 mg heme/g protein. In some embodiments, the biomass of M. capsulatus has at least 0.01 mg, at least 0.05 mg, at least 0.1 mg, at least 0.5 mg, at least 1.0 mg heme per gram protein in the protein isolate. In certain embodiments, Methylococcus capsulatus protein isolate contains 0.01 to 10 mg heme/g protein, such as 0.05 to 0.1, 0.1 to 1, 1 to 5, 5 to 10, 0.2 to 0.5, 0.5 to 1, 1 to 2, 2 to 4, or 0.05 to 5 mg heme/g protein.

In certain embodiments, Methylococcus capsulatus protein isolate is enriched in one or more of the following amino acids: L-cysteine, L-tryptophane, L-aspartic acid, L-tyrosine, and L-phenylalanine when compared to Methylococcus capsulatus biomass from which it is prepared. For example, in some embodiments, the concentration by weight of one or more of the above-listed amino acids in protein isolate is increased by at least 5%, 10%, 15%, 20%, 25%, 30%, or 35% when compared to the biomass from which it is prepared.

Protein isolate prepared from Methylococcus capsulatus cultured under low copper conditions typically has higher crude protein content than protein isolate prepared in the same manner from the methanotropic bacterium cultured under normal or high copper conditions. In certain embodiments, the protein isolate prepared from Methylococcus capsulatus cultured under low copper conditions has at least 72%, such as at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, or at least 96% crude protein. In certain embodiments, the protein isolate is composed of about 72% to about 99%, about 75% to about 99%, about 78% to about 99%, about 80% to about 99%, about 82% to about 99%, about 85% to about 99%, about 90% to about 99%, about 72% to about 95%, about 75% to about 95%, about 78% to about 95%, about 80% to about 95%, about 82% to about 95%, about 85% to about 95%, about 90% to about 95%, about 72% to about 90%, about 75% to about 90%, about 78% to about 90%, about 80% to about 90%, about 72% to about 85%, about 75% to about 85%, about 78% to about 85%, about 80% to about 85%, crude protein.

In certain embodiments, protein isolate from Methylococcus capsulatus cultured under low copper conditions is composed of at least 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% true protein. In certain embodiments, protein isolate from Methylococcus capsulatus cultured under low copper conditions is composed of about 62% to about 99%, about 62% to about 95%, about 62% to about 90%, about 62% to about 85%, about 62% to about 80%, about 62% to about 75%, about 65% to about 99%, about 65% to about 95%, about 65% to about 90%, about 65% to about 85%, about 65% to about 80%, about 65% to about 75%, about 70% to about 99%, about 70% to about 95%, about 70% to about 90%, about 70% to about 85%, about 70% to about 80%, about 75% to about 99%, about 75% to about 95%, about 75% to about 90%, about 75% to about 85%, about 80% to about 99%, about 80% to about 95%, or about 80% to about 90% by weight true protein.

Protein isolate prepared from Methylococcus capsulatus cultured under low copper conditions preferably contains minimal amount of nucleic acid to minimize potential advance effects of a high nucleic acid level to animals or human that consume the protein isolate (e.g., causing gout and kidney stones). In certain embodiments, the protein isolate prepared from Methylococcus capsulatus cultured under low copper conditions has at most 10%, 9%, 8%, 7%, preferably, at most 6%, 5%, 4%, 3%, 2% or 1%, by weight nucleic acids.

Protein isolate prepared from Methylococcus capsulatus cultured under low copper conditions preferably contains a minimal amount of ash. In certain embodiments, Methylococcus capsulatus protein isolate has less than about 10%, 9%, 8%, preferably, less than about 7%, 6%, 5%, 4%, 3%, 2%, or 1%, by weight ash.

Protein isolate prepared from Methylococcus capsulatus cultured under low copper conditions preferably contains a minimal amount of fat. In certain embodiments, Methylococcus capsulatus protein isolate has less than about 15%, 14%, 13%, 12%, 11%, preferably less than about 10%, 9%, 8%, 7%, 6%, 5%, 4%, 3%, 2%, or 1%, by weight crude fat.

Protein isolate prepared from Methylococcus capsulatus cultured under low copper conditions preferably contains an increased level of heme. In some embodiments, Methylococcus capsulatus protein isolate has at least 0.01 mg, at least 0.05 mg, at least 0.5 mg, at least 1.0 mg heme per gram protein in the biomass. In certain embodiments, Methylococcus capsulatus protein isolate contains 0.01 to 10 mg heme/g protein, such as 0.01 to 0.1, 0.1 to 1, 1 to 5, 5 to 10, 0.2 to 0.5, 0.5 to 1, 1 to 2, 2 to 4, or 0.05 to 0.5 mg heme/g protein.

In certain embodiments, the protein isolate prepared from Methylococcus capsulatus cultured under low copper conditions has a copper level at most 100, 90, 80, 70, 60, 50, 40, 30, 20, 10, or 5 mg copper per kg protein isolate. In some embodiments, the protein isolate prepared from Methylococcus capsulatus cultured under low copper conditions has a copper level has a copper level from 1 to 100, from 1 to 5, from 5 to 10, from 10 to 20, from 20 to 30, from 30 to 40, from 40 to 50, from 50 to 60, from 60 to 70, from 70 to 80, from 80 to 90, from 90 to 100, from 1 to 90, from 1 to 80, from 1 to 70, from 1 to 60, from 1 to 50, from 1 to 40, from 1 to 30, from 1 to 20, from 1 to 10, from 5 to 90, from 5 to 80, from 5 to 70, from 5 to 60, from 5 to 50, from 5 to 40, from 5 to 30, from 5 to 20, from 10 to 90, from 10 to 80, from 10 to 70, from 10 to 60, from 10 to 50, from 10 to 40, from 10 to 30, from 20 to 90, from 20 to 80, from 20 to 70, from 20 to 60, from 20 to 50, from 20 to 40 mg copper/kg protein isolate.

The Methylococcus capsulatus protein isolate also possesses a protein digestibility corrected amino acid score (PDCAAS) of at least 0.9. In certain embodiments, the Methylococcus capsulatus protein isolate has a PDCAAS score of at least 0.91, 0.92, 0.93, 0.94, 0.95, 0.96, 0.97, 0.98, or 0.99. PDCAAS is a method of evaluating the quality of a protein based on both the presence of essential amino acids for human nutrition and their digestibility. The PDCAAS rating was adopted by the US FDA and the Food and Agricultural Organization of the United Nations/World Health Organization (FAO/WHO) as the preferred best method to determine protein quality.

As disclosed herein, PDCAAS is determined using the Protein Digestibility assay provided by Megazyme (see K-PDCAAS Data, Megazyme, 2019), an in vitro enzyme digestion method with a very high correlation to the traditional in vivo assay using rat subjects to measure the amount of protein digestion that occurs when fed a protein containing product. Using the PDCAAS method, protein quality rankings are determined by comparing the amino acid profile of the protein sample against a standard amino acid profile as well as some digestibility measurement. A higher PDCAAS in general indicates higher protein quality in providing the required amounts of indispensable amino acids. Typically, the PDCAAS are capped at 1.0, 1.0 representing a “perfect” protein. However, in vitro PCAAS measurement can lead to scores greater than 1.0. Animal derived proteins tend to have a higher PDCAAS score (close to 1.0) than most plant-based proteins (typically <0.9) (FIG. 9).

Methylococcus capsulatus protein isolate derived from Methylococcus capsulatus biomass grown on a natural gas-derived substrate are distinctive with respect to their carbon fingerprint as represented by their δ¹³C values (as are the products derived from such Methylococcus capsulatus microorganisms). By way of background, stable isotopic measurements and mass balance approaches are widely used to evaluate global sources and sinks of methane (see Whiticar and Faber, Org. Geochem. 10:759, 1986; Whiticar, Org. Geochem. 16: 531, 1990). To use δ¹³C values of residual methane to determine the amount oxidized, it is necessary to know the degree of isotopic fractionation caused by microbial oxidation of methane. For example, aerobic methanotrophs can metabolize methane through a specific enzyme, methane monoxygenase (MMO). Methanotrophs convert methane to methanol and subsequently formaldehyde. Formaldehyde can be further oxidized to CO₂ to provide energy to the cell in the form of reducing equivalents (NADH), or incorporated into biomass through either the RuMP or Serine cycles (Hanson and Hanson, Microbiol. Rev. 60:439, 1996), which are directly analogous to carbon assimilation pathways in photosynthetic organisms. More specifically, a Type I methanotroph, such as Methylococcus capsulatus, uses the RuMP pathway for biomass synthesis and generates biomass entirely from CH₄, whereas a Type II methanotroph uses the serine pathway that assimilates 50-70% of the cell carbon from CH₄ and 30-50% from C02 (Hanson and Hanson, 1996). Methods for measuring carbon isotope compositions are provided in, for example, Templeton et al. (Geochim. Cosmochim. Acta 70:1739, 2006), which methods are hereby incorporated by reference in their entirety. Example 11 describes the characterization of stable carbon isotope distribution in the cells of different fermentation batches of Methylococcus capsulatus microorganisms. The δ¹³C of the products described herein (i.e., a Methylococcus capsulatus microorganism, related biomass, protein isolate and food compositions derived therefrom) can vary depending on the source and purity of the C₁ substrate used, as demonstrated in Example 11.

In certain embodiments, the Methylococcus capsulatus microorganism of the present disclosure, and related biomass, protein isolate and food compositions derived therefrom, exhibit a δ¹³C of less than −30‰, less than −31‰, less than −32‰, less than −33‰, less than −34‰, less than −35‰, less than −36‰, less than −37‰, less than −38‰, less than −39‰, less than −40‰, less than −41‰, less than −42‰, less than −43‰, less than −44‰, less than −45‰, less than −46‰, less than −47‰, less than −48‰, less than −49‰, less than −50‰, less than −51‰, less than −52‰, less than −53‰, less than −54‰, less than −55‰, less than −56‰, less than −57‰, less than −58‰, less than −59‰, less than −60‰, less than −61‰, less than −62‰, less than −63‰, less than −64‰, less than −65‰, less than −66‰, less than −67‰, less than −68‰, less than −69‰, or less than −70‰.

In certain embodiments, the Methylococcus capsulatus microorganism of the present disclosure, related biomass, and protein isolate and food compositions derived therefrom, exhibit a δ¹³C of about −35‰ to about −50‰, −45‰ to about −35‰, or about −50‰ to about −40‰, or about −45‰ to about −65‰, or about −60‰ to about −70‰, or about −30‰ to about −70‰.

In further embodiments, the Methylococcus capsulatus microorganism of the present disclosure, related biomass, and protein isolate and food compositions derived therefrom has a δ¹³C of less than about −30‰, or ranges from about −40‰ to about −60‰.

Characterization of SVC of some C₁ metabolizing microorganisms including Methylococcus capsulatus, cultivated in the presence of a natural gas-derived feedstock is illustrated in the examples, hereinbelow.

Methylococcus capsulatus microorganisms from which protein isolate is derived may be genetically modified or non-genetically modified. In a preferred embodiment, the Methylococcus capsulatus is non-genetically modified.

In certain embodiments, Methylococcus capsulatus protein isolate is derived from Methylococcus capsulatus (Bath), Methylococcus capsulatus (Texas), Methylococcus capsulatus (Aberdeen), or a combination thereof. In a preferred embodiment, Methylococcus capsulatus protein isolate is derived from Methylococcus capsulatus (Bath).

Methylococcus capsulatus protein isolate may be in the form of a liquid, gel, powder, grains, flakes, chunks, nuggets, slurry, or crumb.

In certain embodiments, the Methylococcus capsulatus protein isolate is processed under good manufacturing practice (GMP) conditions.

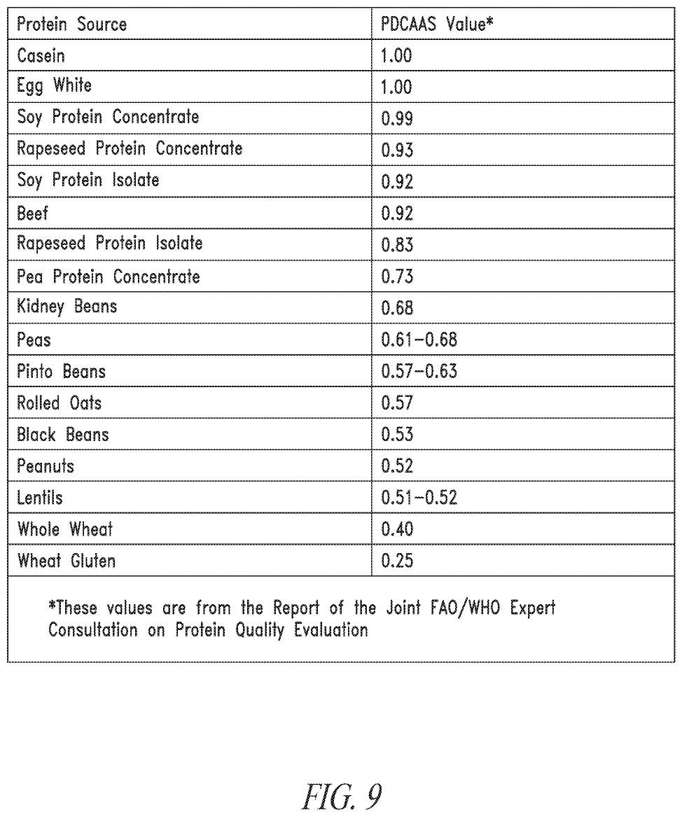

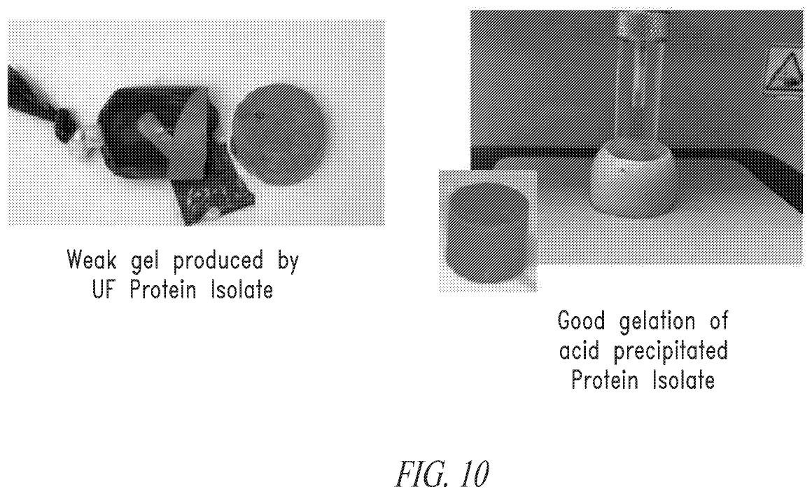

Methylococcus capsulatus protein isolate possess desirable functionality for incorporation into food compositions. Methylococcus capsulatus protein isolate prepared with acid precipitation as described in Example 4 demonstrated good gelation properties (FIGS. 10 and 11). The gelation property of Methylococcus capsulatus protein isolate makes it desirable for use as a protein binder or egg white replacement (e.g., in baked goods, meringue) in a food composition. Gelated foods that Methylococcus capsulatus protein isolate can be used in include meat-substitutes, dairy-free yogurt, protein pudding, protein gels, chips, extruded snacks, pasta, fish/seafood substitutes (e.g., paste, sticks, filets), and surimi-substitute. In certain embodiments, a 15% solution of Methylococcus capsulatus protein isolate that has been heated for 75° C. for one hour has a gel strength of about 10 g to about 300 g (e.g., about 15 g to about 300 g, about 25 g to about 300 g, about 50 g to about 300 g, about 15 g to about 150 g, or about 25 g to about 250 g). In certain embodiments, a 15% solution of Methylococcus capsulatus protein isolate that has been heated for 75° C. for one hour has a gel strength of about 10 g, 20 g, 30 g, 40 g, 50 g, 60 g, 70 g, 80 g, 90 g, 100 g, 110 g, 120 g, 130 g, 140 g, 150 g, 160 g, 170 g, 180 g, 190 g, 200 g, 210 g, 220 g, 230 g, 240 g, 250 g, 260 g, 270 g, 280 g, 290 g, or 300 g, or any range between two of the above values. In preferred embodiments, a 15% solution of Methylococcus capsulatus protein isolate that has been heated for 75° C. for one hour has a gel strength of about 250 g to about 300 g. A Brookfield CT3 Texture Analyzer may be used to perform compression testing of the gel.

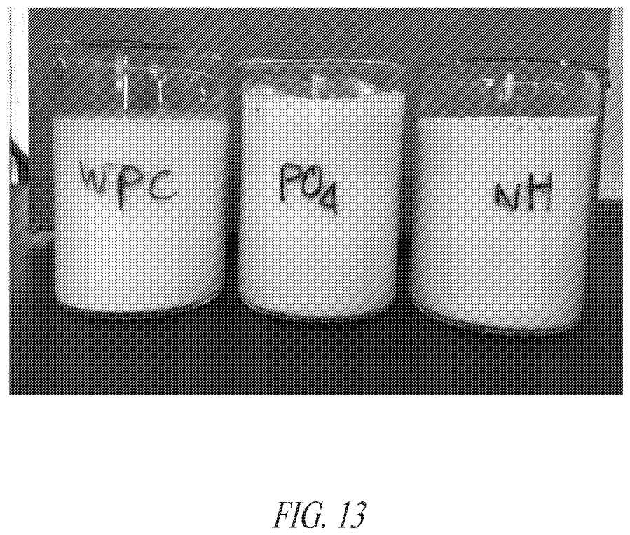

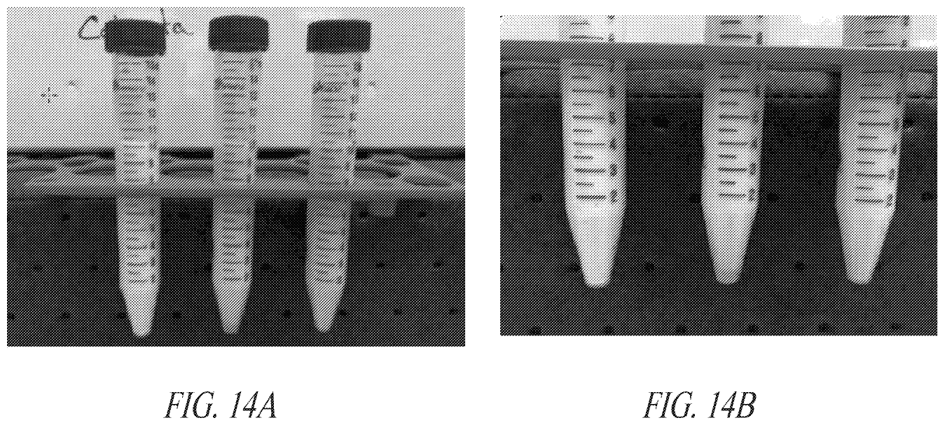

Methylococcus capsulatus protein isolate can also form stable emulsions, particularly with the addition of an emulsifying agent or in the absence of heat treatment step prior to making the emulsion (e.g., with a homogenizer, microfluidizer, or mixer) (FIGS. 13-14B). In certain embodiments, Methylococcus capsulatus protein isolate is mixed with an oil, preferably a plant-based oil, to form an emulsion. In a further embodiment, an emulsion comprising Methylococcus capsulatus protein isolate comprises about 2% to about 12% Methylococcus capsulatus protein isolate and about 3% to about 25% oil. In yet a further embodiment, an emulsion comprising Methylococcus capsulatus protein isolate comprises about 2% to about 5% Methylococcus capsulatus protein isolate and about 12% to about 18% oil. Emulsion based foods that Methylococcus capsulatus protein isolate can be used for include dressings, dips, spreads, mayonnaise, Hollandiase sauce, vinaigrettes, dairy-free coffee creamer, creamy sauces, creamy soups, butter, margarine, protein shakes, and nutrition beverages. In certain embodiments, an emulsion comprising Methylococcus capsulatus protein isolate is stable during storage at room temperature without phase separation or forming a creamy layer for at least 24 hours, such as at least 36 hours, 48 hours, 60 hours, or 72 hours.

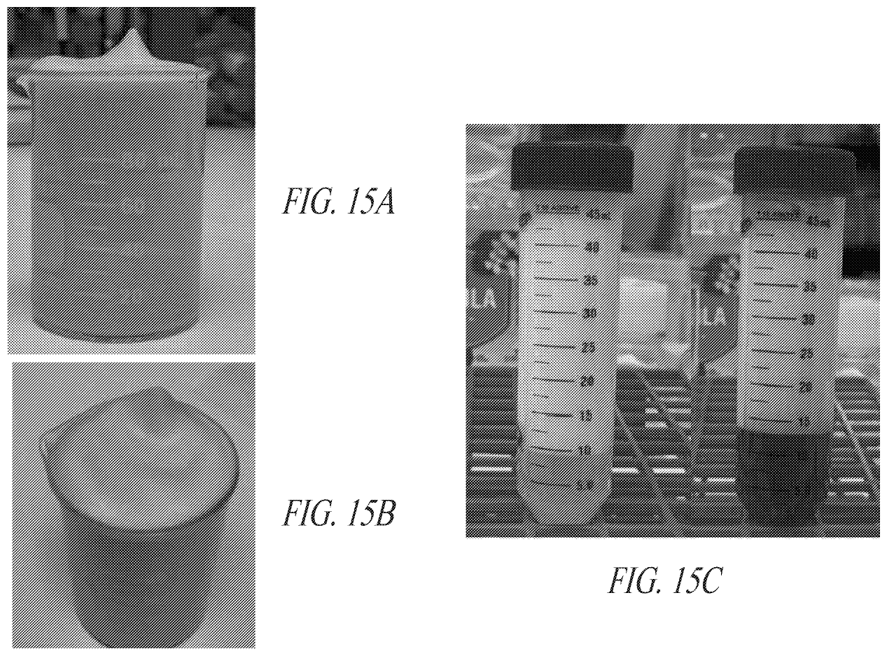

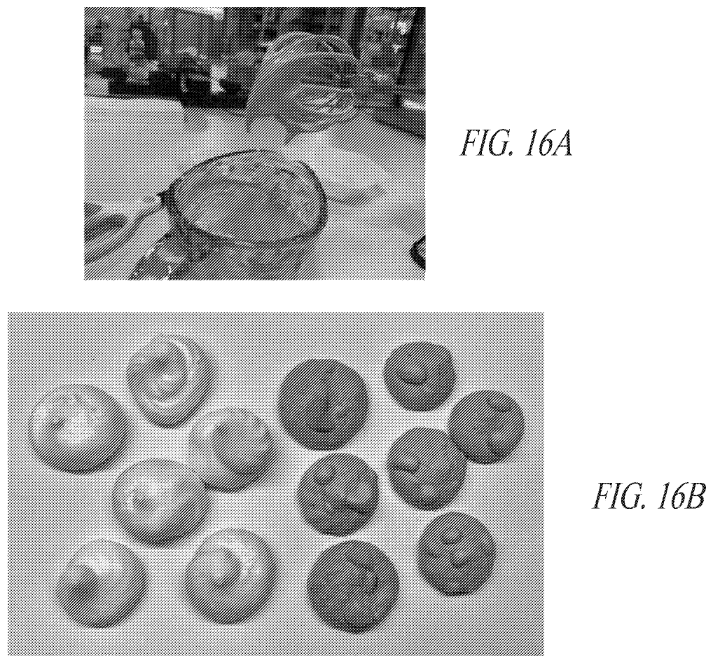

Methylococcus capsulatus protein isolate also exhibits good foaming (FIGS. 15-16 and Table 3), which would be desirable for use as an egg white substitute (e.g., in baked goods, meringue) and in meat-substitutes and chips. Exemplary baked goods include meringue, crackers, cake (e.g., angel food cake), pies, pastries, cookies brownies, buns, bread, and quick bread. In certain embodiments, foam produced from Methylococcus capsulatus protein isolate has a foam overrun of at least 200%, 225%, 250%, 275%, 300%, 325%, 350%, 375%, 400%, 425%, 450%, 475%, 500%, 525%, 550%, 575%, or 600%. In further embodiments, Methylococcus capsulatus protein isolate has a foam overrun of about 200% to about 600%, about 250% to about 600%, about 300% to about 600%, about 400% to about 600%, or about 500% to about 600%. % foam overrun=(Weight of solution−weight of foam)/(weight of foam)×100% and may be measured using a pycnometer having 11.5 ml capacity (Cole-Parmer Part #EW-38001-00). In certain embodiments, Methylococcus capsulatus protein isolate has a foam stability of at least 30 minutes, 35 minutes, 40 minutes, 45 minutes, 50 minutes, 55 minutes, or 1 hour. Foam stability may be measured by: foam stability (%)=(residual foam volume)/(total foam volume)×100.

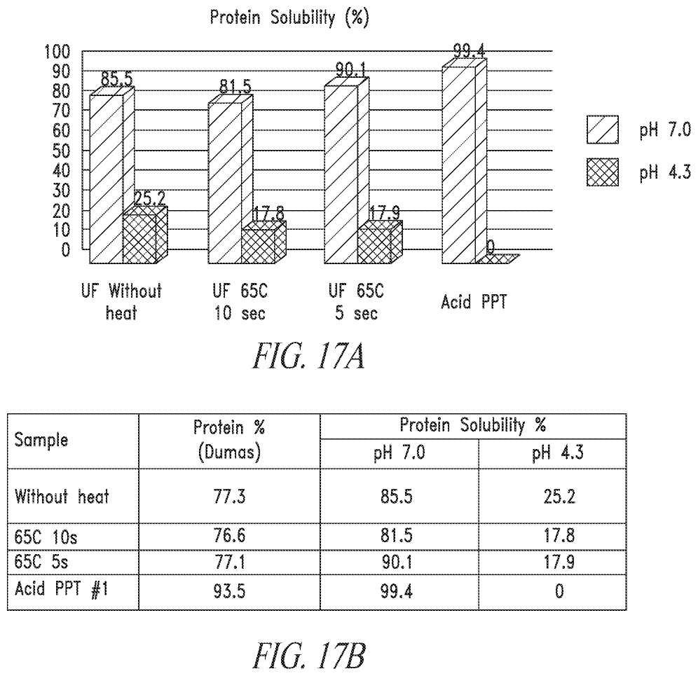

Methylococcus capsulatus protein isolate also exhibits good solubility, particularly at neutral pH (˜pH 7.0) (FIGS. 17A-17B). Exemplary food compositions where solubility is desirable include sauces, dips, dressings, soups, spreads, protein shakes, nutrition beverages, protein gels, dairy-free products (e.g., creamer, ice cream, milk), and protein powders. In certain embodiments, Methylococcus capsulatus protein isolate has at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% solubility at neutral pH. Solubility may be measured by dissolving Methylococcus capsulatus protein isolate in water to prepare a 2% solution and centrifuging at 7,000 rpm (4000×g) for 10 minutes and the crude protein content of the supernatant measured by the Dumas method.

The term “whole cell product,” “whole cell ingredient,” “whole cell protein,” “whole cell proteins,” “whole cell preparation” or the like as used herein refers to a product made from Methylococcus capsulatus biomass without undergoing a step of separating soluble proteins from other components of the biomass, especially solid components such as cell debris and/or cell wall. In certain embodiments, the process of producing a whole cell product may include a heating step to activate native nucleases (e.g., native endonucleases) to degrade some of the nucleic acids (DNA and RNA) present in the biomass.

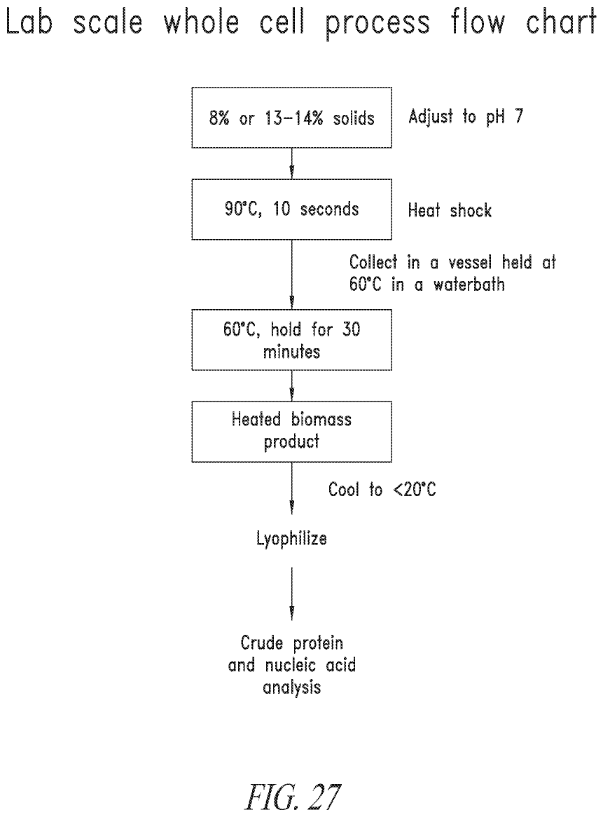

For example, whole cell products may be produced by re-suspending harvested Methylococcus capsulatus biomass in a liquid (e.g., water), optionally subjecting the re-suspension to heat shock (e.g., at 90° C. for 10 seconds) to activate native nucleases (e.g., native endonucleases) in the biomass, heating the re-suspension (e.g., at 60° C. for 30 minutes) to allow for reactions catalyzed by the activated nucleases, cooling the heated re-suspension (e.g., to less than 20° C.), and lyophilizing the cooled composition. In certain embodiments, the process may include a step of homogenization, preferably after the heating step. An exemplary process of preparing whole cell products is described in Example 16, a flow chart of which is shown in FIG. 27. In certain embodiments, the Methylococcus capsulatus whole cell products are produced under good manufacturing practice (GMP) conditions.

The whole cell products provided herein may have crude protein, true protein, nucleic acid, ash, crude fat, heme or heme iron amounts, and levels of enrichment of one or more amino acids (e.g., L-cysteine, L-tryptophane, L-aspartic acid, L-tyrosine, L-phenylalanine) similar to those of Methylococcus capsulatus protein isolates described above. In certain embodiments, the whole cell products have certain desirable functionalities (e.g., water and oil binding ability) for incorporation into food compositions.

### Food Compositions

Methylococcus capsulatus protein isolate can be used for a variety of food compositions suitable for human consumption, meaning that they can be consumed by humans without ill health effects and provide caloric benefit.

Examples of food compositions that Methylococcus capsulatus protein isolate can be used in preparation of include meat substitutes (e.g., patty, meatball, crumble, sausage, jerky, loaf, filet, bacon, hot dog, or nugget), fish/seafood substitutes (e.g., surimi, paste, fish, filet), dairy substitutes (e.g., non-dairy milk, non-dairy creamer, non-dairy cream, non-dairy yogurt, non-dairy whipped topping, and non-dairy ice cream), baked goods (e.g., meringue, crackers, cake (e.g., angel food cake), pies, pastries, cookies brownies, buns, bread, and quick bread), sauces (e.g., Hollandaise sauce), dressings, dips, spreads, mayonnaise, vinaigrettes, creamy sauces, creamy soups, butter, margarine, protein bars, extruded protein crisps, protein shakes, protein gels, and nutrition beverages. Methylococcus capsulatus protein isolate can be used in protein fortified foods (e.g., protein fortified snacks, chips, crackers, puffs, and pasta).

An exemplary emulsion composition comprising Methylococcus capsulatus protein isolate comprises about 2-6% Methylococcus capsulatus protein isolate, 12-18% oil (e.g., plant-based oil), and optionally, 0.1-0.75% emulsifying agent (e.g., sodium hexamethaphosphate, DATEM, or both).

An exemplary meringue composition comprising Methylococcus capsulatus protein isolate comprises about 15-18 g water, 3-18 g Methylococcus capsulatus protein isolate, 10-20 g sweetener (e.g., sugar), and 0.3-1.0 g cream of tartar (potassium bitartrate). Another exemplary meringue composition comprises: 18 g water, 5 g Methylococcus capsulatus protein isolate, 15 g sugar, and 0.4 gram cream of tartar, which makes approximately 10 meringue cookies.

An exemplary cracker composition comprising Methylococcus capsulatus protein isolate comprises about 5-20% Methylococcus capsulatus protein isolate, about 55-75% flour (e.g., wheat flour), 0.94% leavening agent (e.g., sodium bicarbonate, ammonium bicarbonate, or both), about 5-10% sweetener (e.g., sugar or corn syrup), 0.55% salt, about 0.5-2% emulsifying agent (e.g., Dimodan), and 2.2-6.6% oil. Another exemplary cracker composition comprising Methylococcus capsulatus protein isolate comprises 16 g wheat flour, 3.75 g Methylococcus capsulatus protein isolate, 0.15 g sodium bicarbonate, 1.35 g sugar, 0.13 g salt, 0.24 g dimodan (emulsifier), 0.08 g aluminum bicarbonate, and 1.1 g vegetable oil, which makes approximately 12 small crackers.

The heme proteins found in Methylococcus capsulatus protein isolate make it particularly suitable for use in meat-substitutes. The heme proteins provide the uncooked meat-substitutes a natural fresh meat color, which darkens during cooking, cooking aroma, and slight metallic taste without necessitating the use of genetically modified organisms.

As disclosed in more detail below, depending on final food products, food compositions provided herein may comprise Methylococcus capsulatus protein isolate in an amount of about 0.5% to about 35% by weight, such as 0.5% to 5%, 5% to 10%, 10% to 15%, 15% to 20%, 20% to 25%, 25% to 30%, 30% to 35%, 1% to 10%, 10% to 20%, 20% to 35%, 1% to 15%, 15% to 35%, 1% to 20%, 1% to 35%, 2% to 5%, 2% to 10%, 2% to 15%, 2% to 20%, 2% to 25%, 2% to 30%, 2% to 35%, 5% to 10%, 5% to 15%, 5% to 20%, 5% to 25%, 5% to 30%, 5% to 35%, 10% to 25%, 10% to 35%, 15% to 25%, or 15% to 35%.

In certain embodiments, a meat-substitute patty (hamburger) comprises about 2.5-5 g (13-25%) Methylococcus capsulatus protein isolate, about 12-20 g (50-70%) plant based protein (e.g., textured soy protein), about 2.5-4.5 g (5-15%) oil (e.g., plant-based oil), and optionally water, about 0-4 g (0-11%) a binder (e.g., flour), at least one spice (about 1.48-2.18%), or a combination thereof. In certain embodiments, the Methylococcus capsulatus protein isolate is acid precipitated during processing from the biomass.

An exemplary meat-substitute patty (hamburger) composition comprising Methylococcus capsulatus protein isolate comprises 2 g flour, 2.5 g Methylococcus capsulatus protein isolate, and 15 g texturized soy. In certain embodiments, the Methylococcus capsulatus protein isolate is acid precipitated during processing from the biomass.

Another exemplary meat-substitute patty (hamburger) composition comprising Methylococcus capsulatus protein isolate comprises 2 g flour, 3.5 g Methylococcus capsulatus protein isolate, 1.2 g coconut oil, 0.2 g salt, 0.15 g garlic powder, 0.15 g onion powder, and 15 g soaked texturized soy, for a total pre-cooked weight of 22.20 g. In certain embodiments, the Methylococcus capsulatus protein isolate is acid precipitated during processing from the biomass.

Yet another exemplary meat-substitute patty (hamburger) composition comprising Methylococcus capsulatus protein isolate comprises 2 g flour, 4.5 g Methylococcus capsulatus protein isolate, 1.2 g coconut oil, 0.2 g salt, 0.15 g garlic powder, 0.15 g onion powder, and 15 g soaked texturized soy, for a total pre-cooked weight of 23.20 g. In certain embodiments, the Methylococcus capsulatus protein isolate is acid precipitated during processing from the biomass.

Yet another exemplary meat-substitute patty (hamburger) composition comprising Methylococcus capsulatus protein isolate comprises 9% flour, 15.7% Methylococcus capsulatus protein isolate, 5.4% coconut oil, 0.9% salt, 0.7% garlic powder, 0.7% onion powder, and 67.6% hydrated textured soy. In certain embodiments, the Methylococcus capsulatus protein isolate is acid precipitated during processing from the biomass.

An exemplary protein fortified chip composition comprises 19-25 g (65-80%) grain flour (e.g., corn flour, rice flour, quinoa, wheat), 6.5-8 g (20-26%) Methylococcus capsulatus protein isolate, 0.3-0.5% leavening agent, and optionally water. Another exemplary protein fortified chip composition comprising Methylococcus capsulatus protein isolate comprises 22.9 g masa, 7.1 g Methylococcus capsulatus protein isolate, 0.45 g Aquamin™ (CaCO₃), and 26.5 g water.

In certain embodiments, a dairy-free ice cream comprises 3-10% Methylococcus capsulatus protein isolate, 45-60% water, 10-20% sweetener (e.g., sugar), 8-16% oil (e.g., plant-based oil such as coconut oil), and optionally 10-30% fruit, filling, flavoring or a combination thereof.

An exemplary dairy-free strawberry ice cream composition comprising Methylococcus capsulatus protein isolate comprises 4 g Methylococcus capsulatus protein isolate, 50 g water, 7.7 g coconut oil, 16.2 g sugar, 0.125 g citric acid, 11.1 g strawberry puree, 11.1 g strawberries per 100 g batch of ice cream.

An exemplary dairy-free chocolate ice cream comprises 45-60% water, 3-10% Methylococcus capsulatus protein isolate, 8-16% oil (e.g., plant-based oil such as coconut oil), 10-25% sweetener, and 6.5-8% cocoa powder.

Another exemplary chocolate dairy-free ice cream composition comprising Methylococcus capsulatus protein isolate comprises 4.78 g Methylococcus capsulatus protein isolate, 59.7 g water, 10.6 g coconut oil, 21.1 g sugar, 3.8 g cocoa powder per 100 g batch of ice cream.

Another exemplary chocolate dairy-free ice cream composition comprising Methylococcus capsulatus protein isolate comprises 3.99 g Methylococcus capsulatus protein isolate, 55.29 g water, 13.7 g coconut oil, 21.42 g sugar, 3.74 g cocoa powder, and 1.87 g dark cocoa powder per 100 g batch of ice cream.

In certain embodiments, a food composition comprising Methylococcus capsulatus protein isolate has a reddish color. In other embodiments, a food composition comprising Methylococcus capsulatus protein isolate does not have a reddish color.

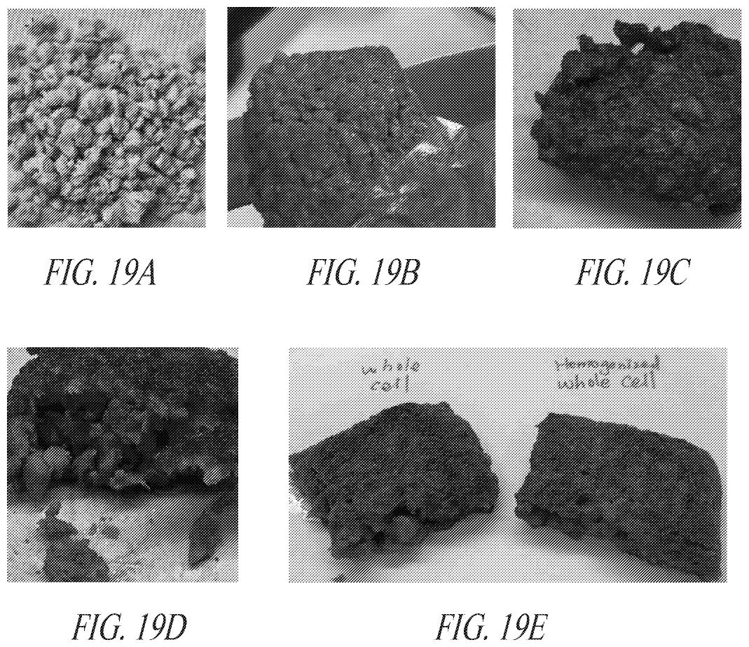

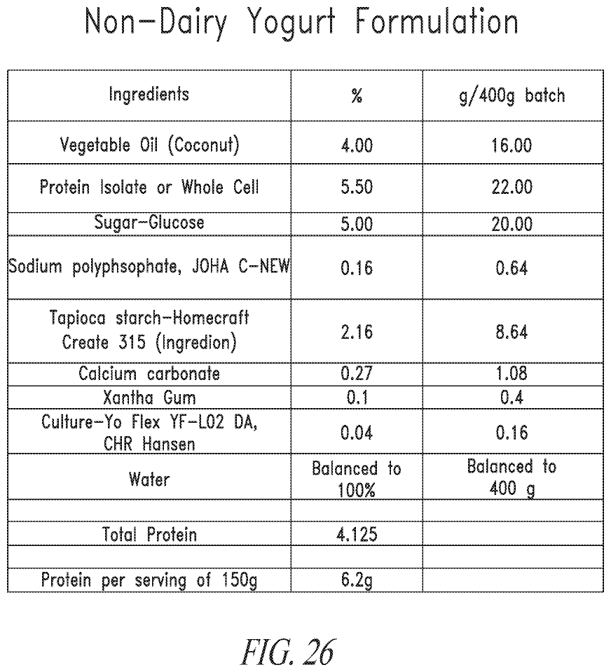

In addition to food compositions made from Methylococcus capsulatus protein isolate, the present disclosure also provides food compositions made from Methylococcus capsulatus whole cell products. For example, whole cell products may be used to prepare meat substitute compositions (e.g., meat substitute patty) and non-diary yogurt. Exemplary meat substitute compositions and non-diary yogurt comprising Methylococcus capsulatus whole cell products are described in Examples 17 and 18 (see FIGS. 19E and 26).

### Additives and Additional Ingredients to Food Compositions

One or more food additives may be combined with Methylococcus capsulatus protein isolate or whole cell product in the food compositions described herein. A food additive is any substance that is added to the food composition, which may affect the characteristics of the food composition.

In certain embodiments, an emulsifying agent, also known as an emulsifier or emulgent, is added to the Methylococcus capsulatus protein isolate or whole cell product or food composition made therefrom. Emulsifying agents may be particularly useful in food compositions that are emulsions. An emulsifying agent is a surface-active agent that acts as a border between two immiscible liquids such as oil and water, allowing them to be blended into stable emulsions. Emulsifiers also reduce stickiness, control crystallization and prevent separation. Emulsifying agents are compounds that typically have a polar or hydrophilic (i.e., water-soluble) part and a non-polar (i.e., hydrophobic or lipophilic) part. Because of this, emulsifiers tend to have more or less solubility either in water or in oil. Emulsifiers that are more soluble in water (and conversely, less soluble in oil) will generally form oil-in-water emulsions, while emulsifiers that are more soluble in oil will form water-in-oil emulsions.

Examples of emulsifying agents that can be used as food additives include a phosphate salt, lecithin, diacetyl tartaric acid ester of monoglyceride (DATEM), acetic acid ester of monoglyceride (AMG), lactic acid ester of monoglyceride (LMG), citric acid ester of monoglyceride (CMG), succinic acid ester of monoglyceride (SMG), sodium stearoyl lactylate, monoglyceride, diglyceride, polysorbate, carrageenan, guar gum, calcium stearoyl lactylate (CSL), polyglycerol ester (PGE), polyglycerol polyricinoleate (PGPR), sorbitan ester (SOE), sorbitan monostearate, propylene glycol ester of fatty acid (PGME), sugar ester (SE), dimodan, or any combination thereof. Examples of phosphate salts used in food production include sodium phosphate, potassium phosphate, calcium phosphate, sodium hexameta phosphate, and salts of orthophosphoric acid diphosphate, triphosphate, and polyphosphate.

In certain embodiments, Methylococcus capsulatus protein isolate or whole cell product or food composition made therefrom comprises a viscosity increasing agent, also known as thickener or gelling agent. A viscosity increasing agent increases viscosity and improves stability by preventing emulsions from separating, ice crystals from forming and ingredients from settling. Viscosity increasing agents include starches (such as arrowroot starch cornstarch, potato starch, and tapioca), whey, vegetable gums (such as guar gum, locust bean gum, xanthan gum), collagen, gelatin, alginic acid, calcium alginate, potassium alginate, calcium alginate, agar-agar, carrageenan, pectin, or any combination thereof.

In certain embodiments, Methylococcus capsulatus protein isolate or whole cell product or food composition made therefrom comprises a binder. Binders include flour (e.g., wheat, rice, tapioca, konjac, rye, acorn, almond, amaranth, bean, cassava, banana, chestnut, chickpea, corn, hemp, mequite, pea, sorghum, soybean, arrowroot, taro, quinoa, manioc, and teff), starches (e.g., corn, rice, potato, tapioca, arrowroot, wheat, sweet potato, sago, and mung bean), gums (e.g., xanthan, guar, agar, locust bean), carrageenan, carboxymethyl cellulose, cellulose, hydroxypropyl methylcellulose, methylcellulose, canola protein, pectin, oat fiber, rice bran, flax seeds, chia seeds, psyllium husk, or any combination thereof.

In certain embodiments, Methylococcus capsulatus protein isolate or whole cell product or food composition made therefrom comprises an anti-caking agent to prevent lumping, caking, or sticking. Examples of anticaking agents include calcium phosphates, silicon dioxide, iron ammonium citrate, silicates (calcium, aluminum and tricalcium) and stearic acid.

In certain embodiments, Methylococcus capsulatus protein isolate or whole cell product or food composition made therefrom comprises a preservative to preserve the natural characteristics of the food, to preserve the appearance of the food, or to increase the shelf value of food for storage. Exemplary preservatives that may be used in Methylococcus capsulatus protein isolate or food composition made therefrom include salt, sugar, alcohol, vinegar, benzoates (e.g., sodium benzoate, benzoic acid), nitrates (e.g., sodium nitrite), sulphites (e.g., sulfur dioxide), sorbates (e.g., sodium sorbate, potassium sorbate), antioxidants (e.g., vitamin C, vitamin E, butylated hydroxyanisole (BHA), butylated hydroxytoluene (BHT)), citric acid, sulfur dioxide, propionic acid, calcium propionate, sodium erythorbate, EDTA, Sodium Erythorbate, Erythorbic Acid, Sodium Diacetate, Sodium Succinate, Grape Seed Extract, Pine Bark Extract, Apple Extract Tea Proplyphenols, Succinic Acid and Ascorbic Acid, parabens, sodium dehydro acetate, and any combination thereof.

In certain embodiments, Methylococcus capsulatus protein isolate or whole cell product or food composition made therefrom comprises a sweetener. Exemplary sweeteners include natural and synthetic sweeteners, such as sucrose (sugar), glucose, fructose, sorbitol, mannitol, corn syrup, high fructose corn syrup, saccharin, aspartame, sucralose, acesulfame potassium (acesulfame-K), honey, agave, molasses, and neotame.

In certain embodiments, Methylococcus capsulatus protein isolate or whole cell product or food composition made therefrom comprises extracts or natural flavorings.

In certain embodiments, Methylococcus capsulatus protein isolate or whole cell product or food composition made therefrom comprises a color additive. Exemplary color additives may be natural or synthetic, such as FD&C Blue Nos. 1 and 2, FD&C Green No. 3, FD&C Red Nos. 3 and 40, FD&C Yellow Nos. 5 and 6, Orange B, Citrus Red No. 2, annatto extract, beta-carotene, grape skin extract, cochineal extract or carmine, paprika oleoresin, caramel color, fruit and vegetable juices, and saffron.

In certain embodiments, Methylococcus capsulatus protein isolate or whole cell product or food composition made therefrom comprises a flavor enhancer, such as monosodium glutamate (MSG), hydrolyzed soy protein, autolyzed yeast extract, disodium guanylate or inosinate.

In certain embodiments, Methylococcus capsulatus protein isolate or whole cell product or food composition made therefrom comprises a fat replacement, such as olestra, cellulose gel, carrageenan, polydextrose, modified food starch, microparticulated egg white protein, guar gum, xanthan gum, whey protein concentrate.

In certain embodiments, Methylococcus capsulatus protein isolate or whole cell product or food composition made therefrom comprises a pH control agent, such as lactic acid, citric acid, ammonium hydroxide, and sodium carbonate.

In certain embodiments, Methylococcus capsulatus protein isolate or whole cell product or food composition made therefrom comprises a leavening agent. Exemplary leavening agents include baking soda (sodium bicarbonate), ammonium bicarbonate, monocalcium phosphate, and calcium carbonate. In certain embodiments, a leavening agent comprises Aquamin, which is a red seaweed-derived multi-mineral complex.

In certain embodiments, Methylococcus capsulatus protein isolate or whole cell product or food composition made therefrom comprises a humectant, such as glycerin or sorbitol.

In certain embodiments, Methylococcus capsulatus protein isolate or whole cell product or food composition made therefrom comprises a nutrient, such as thiamine hydrochloride, riboflavin (Vitamin B2), niacin, niacinamide, folate or folic acid, beta carotene, potassium iodide, iron or ferrous sulfate, alpha tocopherols, ascorbic acid, or Vitamin D.

In certain embodiments, Methylococcus capsulatus protein isolate or whole cell product or food composition made therefrom preferably has a meat-like flavor, and may comprise one or more sulfur-containing amino acids that enhance the flavor when cooked (e.g., cysteine, cysteine, thiamine, and methionine) and/or heme-protective or stabilizing agents (e.g., niacin, imidazone, 4-methyl imidazone, and histidine).

Methylococcus capsulatus protein isolate or whole cell product may be combined with one or more other edible ingredients, including but not limited to grains, fruits, vegetables, proteins, oils, fats, herbs, spices, meats, eggs, and dairy. Edible ingredients may be raw, cooked, natural, synthetic, processed, or unprocessed. Examples of herbs include basil, bay leaves, celery flakes, chervil, cilantro, curry leaves, dill, chives, fenugreek, epazote, rose petals, kaffir lime leaves, lavender, lemongrass, marjoram, mint, oregano, parsley, rosemary, sage, savory, tarragon, and thyme. Examples of spices include salt, pepper, cacao, cocoa powder, caraway seed, cardamom, celery seed, chile powder, chile flakes, cinnamon, citrus zest, cloves, coriander, cumin, dill seed, extracts, fennel, garlic powder, onion powder, ginger powder, horseradish powder, juniper berries, nutmeg, mustard, paprika, poppy seed, porcini powder, saffron, sumac, star anise, turmeric, and vanilla.

In certain embodiments, the Methylococcus capsulatus protein isolate or whole cell product is combined with an additional protein source, including animal based protein, plant based protein, and microorganism based protein. Examples of plant based proteins include soy, pea, rapeseed, beans (e.g., kidney, pinto, black, lentils), nuts, milk, whey, rice, wheat, and oat. Plant based proteins include hydrated and dehydrated plant based proteins. In certain embodiments, a plant based protein is a textured vegetable protein, such as textured soy protein. Examples of animal based proteins include meat, egg, and milk.

In certain embodiments, the Methylococcus capsulatus protein isolate or whole cell product is combined with an animal based protein source. The ratio of the Methylococcus capsulatus protein isolate or whole cell product to the animal based protein source by weight or the ratio of proteins from the Methylococcus capsulatus protein isolate or whole cell product to proteins from the animal based protein source by weight may be from 1:100 to 100:1, such as from 1:20 to 20:1, 1:10 to 10:1, 1:5 to 5:1, 1:3 to 3:1, or 1:2 to 2:1.

In certain embodiments, the Methylococcus capsulatus protein isolate or whole cell product is combined with a plant based protein source. The ratio of the Methylococcus capsulatus protein isolate or whole cell product to the plant based protein source by weight or the ratio of proteins from the Methylococcus capsulatus protein isolate or whole cell product to proteins from the plant based protein source by weight may be from 1:100 to 100:1, such as from 1:20 to 20:1, 1:10 to 10:1, 1:5 to 5:1, 1:3 to 3:1, or 1:2 to 2:1.

In certain embodiments, the Methylococcus capsulatus protein isolate or whole cell product is combined with an oil. As used herein, “oil” means any triacylglyceride derived from plants, animals, or microorganisms that is generally liquid at ordinary room temperature and pressure. Preferably, the oil is a plant based oil, such as soy, rapeseed, canola, palm, palm kernel, coconut, corn, olive, sunflower, cotton seed, cuphea, peanut, camelina sativa, mustard seed, cashew nut, oats, lupine, kenaf, calendula, hemp, coffee, linseed, hazelnut, euphorbia, pumpkin seed, coriander, camellia, sesame, safflower, rice, tung oil tree, cocoa, copra, pium poppy, castor beans, pecan, jojoba, jatropha, macadamia, Brazil nuts, or avocado oil.

It is understood that a food additive may have one or more functions in the food composition. Furthermore, it is contemplated that food compositions provided herein may comprise any combination of additives or ingredients disclosed herein.

The present disclosure also provides methods of making a food composition for human consumption comprising: combining protein isolate or whole cell product of Methylococcus capsulatus, wherein Methylococcus capsulatus is cultured and processed under GMP conditions, with at least one other edible ingredient or food additive. In certain embodiments, the food composition is combined with at least one plant-based protein source, at least one animal-based protein source, and/or at least one bacteria-based protein source. In certain embodiments, the Methylococcus capsulatus protein isolate or whole cell product is used as a meat-based protein substitute, an egg-substitute, or a dairy ingredient substitute in the food composition.

### EXAMPLES

### Example 1

### Growth of Methylococcus capsulatus Bath with Varying Copper Concentration in a Continuous Culture System

The wild-type M. capsulatus Bath was grown in continuous fermentation in 2 L vessels. Nutrients required for growth, except varying amounts of copper, were provided in excess with nutrient master mix feed (MMF). The composition of MMF is shown in Table 1.

TABLE 1
Composition of Master Mix Feed

| | | Concen- | | | |
|---|---|---|---|---|---|
| Material | Source | tration | Units | Material | Units |
| H₃PO₄ | Stock | 85 | % (w/w) | 0.948 | g |
| MgSO₄•7H₂O | Salt | 100 | % | 0.456 | g |
| K₂SO₄ | Salt | 100 | % | 0.201 | g |
| FeSO₄•7H₂O | Salt | 100 | % | 0.025 | g |
| ZnSO₄•7H₂O | Sol-n | 6 | g/L | 0.0264 | mL |
| MnSO₄•H₂O | Sol-n | 2 | g/L | 0.0051 | mL |
| CoSO₄•7H₂O | Sol-n | 2 | g/L | 0.0114 | mL |
| Na₂MoO₄•2H₂O | Sol-n | 2 | g/L | 0.0201 | mL |
| NiCl₂•6H₂O | Sol-n | 2 | g/L | 0.0055 | mL |
| DI Water QS | | | | 1000 | mL |

M. capsulatus Bath has ability to uptake copper into the cell, then use or store all provided copper in concentrations within ranges tested in Example 2 below. To determine the impact of copper concentration on crude protein, copper was fed by a syringe pump at calculated feed rates. The calculation was based on the assumption, that all copper fed, was consumed by the bacteria. For example: for a low copper concentration of 50 μg Cu/g of DCW (dry cell weight) and harvest rate 5 g/L/h of DCW, Cu—CuSO₄·5H₂O feed should be 250 μg/L/h.

Conditions for the methane continuous fermentation are provided in Table 2.

TABLE 2
Parameters for continuous culture with methane

| Parameter | Conditions/Comment |
|---|---|
| Working volume | 1.5 L |
| Temperature | 42° C. |
| Agitation | 1200 RPM |
| Micro-sparger, (20 μM) | Methane |
| Methane flow | 100 mL/min |
| Ring sparger | Air |
| Air Flow to control pO₂ | 360-720 mL/min |
| pO₂ set point | 10% by Air flow |
| pH set point | 6.5 |
| pH control | 1N NaOH, 0.5M H₂SO₄ |
| Master Mix Feed (MMF) | No copper addition |
| MMF Power | Supports growth up to 15 g/L of DCW |
| Nitrogen feed | 0.5M HNO₃ |
| N—NO₃ range | 5-60 mg/L |
| Dilution Rate | 0.1 1/h |

Biomass Collection

Throughout the experiment, wash-out periods were applied after change of condition to wash out biomass obtained at the previous copper concentration and establish new steady state fermentation. Length of wash out period was 20-24 hours or two fermentation volumes. For each set of conditions, two to three liters of the continuously pumped out fermentation broth were collected, which was a volume to obtain 15-20 grams of dry cell weight biomass. During collection, the fermentation broth was stored in fridge. The collected broth was centrifuged, and the wet cell pellets were stored at −80° C. Next, pellets were lyophilized, and the dry cell biomass was subjected to crude protein and elemental analysis. Gas analysis of methane, oxygen, and carbon dioxide was performed during biomass collection periods.

### Example 2

### Protein Isolation Using Microfiltration and Ultrafiltration

### Methods and Materials

Fermentation broth was collected from continuous fermentation and centrifuged. The liquid was discarded. Collected biomass was resuspended in cold de-ionized water to 6-8% total solids. The pH of the mixture adjusted to pH 8 using 5N sodium hydroxide. The solution was homogenized using a Microfluidics LM-10 processor set at 22,000 psi or 1300 bar. The solution was homogenized using 1 pass through the processor and kept cool on ice. The pH was readjusted after homogenization from pH 7 to pH 8 using 5N sodium hydroxide.

The homogenized solution was then adjusted to a total solids of 2% with cold water and centrifuged at 3000×g for 5 minutes at 10° C. The supernatant was carefully decanted into a clean beaker and the pellet discarded. The supernatant solution was subjected to microfiltration using a Millepore Pellicon 2 system and a Durapore 0.65 um filter cassette. The retentate pressure was maintained at 5 psi. The retentate volume was maintained constant with the addition of water. 2-3 volumes of permeate were collected. The permeate was concentrated 10× via ultrafiltration using a Pellicon 2 10 kDa filter cassette. The retentate was freeze dried using a Columbia International Vacuum Freeze Dryer model FD50-B2A.

The dried protein isolate was characterized by % crude protein using the Dumas method using a LECO 828 nitrogen analyzer. Briefly, a measured sample of dry protein isolate powder was rapidly combusted in a hot furnace under a pure oxygen environment. Released nitrogen in the combustion gas was measured by a thermal conductivity detector using helium as the carrier gas. Moisture in the combustion gas was removed by a thermoelectric cooler. The nitrogen content of the protein isolate sample was determined by comparing the amount of released nitrogen to calibration standards with known nitrogen content. The crude protein content in the protein isolate was calculated by multiplying the nitrogen content of the sample by a nitrogen to protein conversion factor.

The nucleic acid content was determined using a Lucigen Masterpure Complete DNA & RNA Purification Kit MC85200.

A flow chart for the protein isolation process using microfiltration followed with ultrafiltration is shown in FIG. 1B.

Results

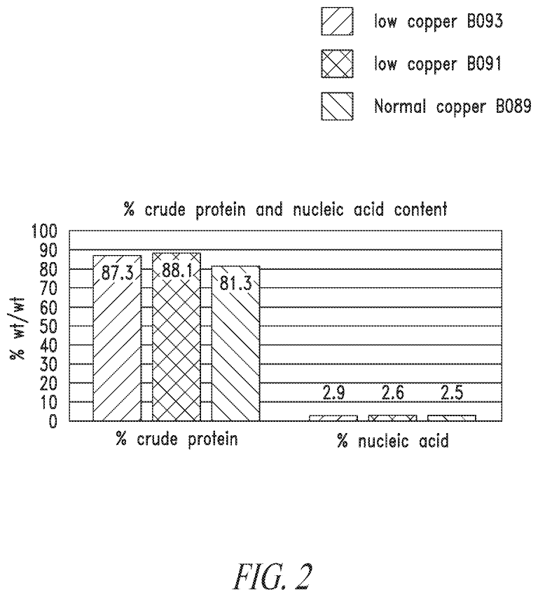

Protein isolate produced from biomass grown under low copper conditions (25 mg copper/kg biomass) was shown to have a higher percentage crude protein of 87-88% compared to protein isolate produced from biomass grown with normal copper levels (154 mg/kg) (see FIG. 2). The nucleic acid content remained low at 2-3% of the total weight of the protein isolate under both low and normal copper conditions.

FIG. 3 shows the increase in crude protein in the biomass collected from fermentations run under a reduced amount of copper. Fermentations under low copper (0.038 g/kg) produced crude protein of 82-83% while fermentations under normal (0.154 g/kg) and high copper (0.371 g/kg) levels produced crude protein of about 75% and 75-77%, respectively.

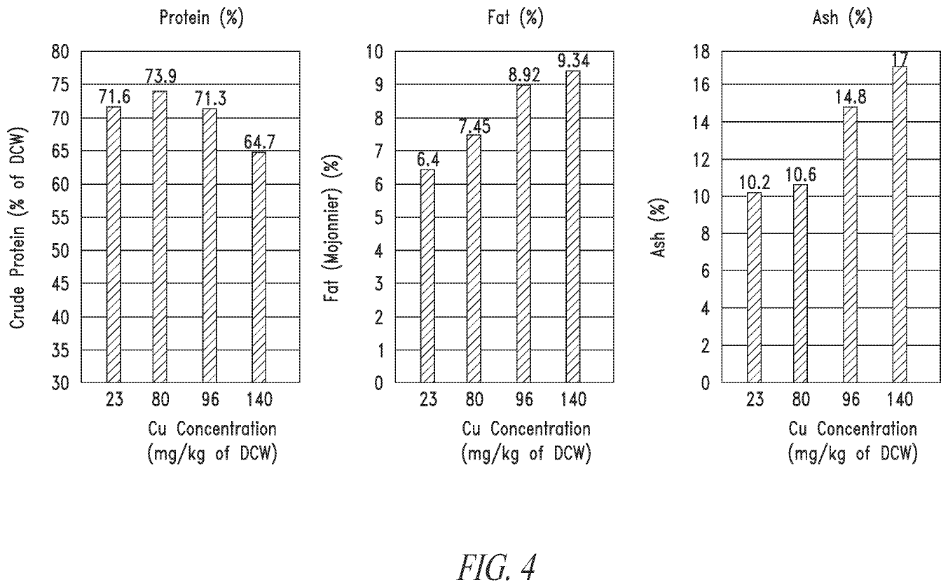

FIG. 4 shows the results of the analysis of crude protein, fat and ash content of the biomass grown under those variable copper conditions (23, 80, 96 and 140 mg/kg). Reduced copper levels increased crude protein percentages of protein isolates while reduced fat and ash percentages.

In summary, the results show higher crude protein percentages from low copper grown biomass compared to normal copper grown biomass (FIGS. 3 and 4). In addition, the higher crude protein of the biomass was maintained in the protein isolate product under the same downstream processing (DSP) conditions (FIG. 2).

### Example 3

### Culturing Methylococcus capsulatus Bath (NCIMB 11132) Under Normal Copper Conditions

The bacteria were cultured at 42° C. in serum bottles containing Higgins minimal nitrate salts medium (NSM) or MM-W1 medium. The headspace composition was adjusted to a 1:1 volume of methane:air. The bottles were shaken at a rate of 200-250 rpm. Alternatively, the culture was maintained on NSM-media plates solidified with 1.5% w/v agar grown in a gas-tight chamber containing a 1:1 (v/v) methane:air gas mixture. Plates were incubated inverted in the chamber at 42° C.

For fermentation, a 3-liter bioreactor containing 1.25 L sterilized media MMF1.1 was inoculated with cells from serum bottle batch cultures (10-20% v/v) grown in the same media supplied with a 1:1 (v/v) mixture of methane and air. The composition of medium MMF1.1 was as follows: 0.8 mM MgSO₄·7H₂O, 40 mM NaNO₃, 0.14 mM CaCl₂), 6 mM NaHCO₃, 4.7 mM KH₂PO₄, 6.8 mM K₂HPO₄, 20.7 μM Na₂MoO₄·2H₂O, 6 μM CuSO₄·5H₂O, 10 μM Fe^{III}—Na-EDTA, and 1 mL per liter of trace metals solution (containing per liter 500 mg FeSO₄·7H₂O, 400 mg ZnSO₄·7H₂O, 20 mg MnCl₂·7H₂O, 50 mg CoCl₂·6H₂O, 10 mg NiCl₂·6H₂O, 15 mg H₃BO₃, 250 mg EDTA). Phosphate, bicarbonate, and Fe^{III}—Na-EDTA were added after media was autoclaved and cooled. The reactor contents were stirred with an overhead impeller at a constant 750 rpm. The culture was fed with a constant methane sparging at about 60 to about 200 mL/min, while concentrated oxygen (>85%) was supplied at a variable rate of 15-90 mL/min and the dissolved oxygen level was maintained below 10% (relative to air saturation of the media).

Temperature in the bioreactor was maintained at 44° C. and pH was maintained at 7.0±0.1 using automated addition of 0.5M NaOH and 0.5M HCl, along with additions of copper and iron (5 μM CuSO₄, 5 μM FeSO₄, 10 μM Fe^{III}—Na-EDTA final concentration) to the culture every 3-6 hours (corresponding to an OD₆₀₀ increase of approximately 3-5 OD units after reaching OD 5). Under these conditions, essentially linear growth was observed, with effective biomass generation rate of more than 5 grams dry cell weight per liter per day to an OD₆₀₀ of greater than 10. Culture biomass was harvested by centrifugation, the cells washed once in MM-W1 media and cell pellets were either frozen at −80° C. or used immediately for fractionation of cellular components.

Nutrient depletion was recognized as an issue that could limit the growth yield during fermentation. To avoid limitation of nutrients, mainly nitrogen and phosphate, nutrient feeds composed of 2-fold concentrated MMF1.1 were initiated after culture OD₆₀₀ exceeded 5. The nutrient feed was initiated at dilution rates corresponding to approximately half of the cultures' growth rate to avoid wash-out and to maintain an increase in OD while expanding the culture volume. The bioreactor fermentation was continued according to the above protocol so that multiple cycles of growth and biomass recovery could be carried out during a single fermentation run.

### Example 4

### Production of Methylococcus capsulatus Protein Isolate from Biomass

### Microfiltration and Ultrafiltration—Sample B066

Fermentation broth from continuous fermentation was collected and centrifuged. The supernatant was discarded. Collected biomass was resuspended in cold de-ionized water at 2.65% solids (4.75 L), and the pH of the mixture was adjusted to pH 8 using 5N NaOH. The solution was homogenized using 1 pass on a Microfluidics LM-10 homogenizer set at 22,000 psi or 1300 bar. The cell lysate was kept cool on ice. The homogenized solution was then adjusted to a total solids of 1.4% with cold water and centrifuged at 3000×g for 5 minutes at 10° C. The supernatant was carefully decanted into a clean beaker and the pellet discarded. The supernatant solution was subjected to microfiltration using a Millepore Pellicon 2 system and a Durapore 0.45 μm filter cassette. The retentate pressure was maintained at 5 psi. The retentate volume was maintained constant with the addition of water. 2-3 volumes of permeate were collected. The permeate was concentrated 10× via ultrafiltration using a Pellicon 2 10 kDa filter cassette. The retentate was freeze dried to a dry powder using a Columbia International Vacuum Freeze Dryer model FD50-B2A. The dried protein isolate was characterized by % crude protein using the Dumas method. A flow chart of this process of preparing protein isolate is shown in FIG. 1B.

### Chitosan and Ultrafiltration—Sample B058

Fermentation broth from continuous fermentation was collected and centrifuged. The supernatant was discarded. Collected biomass was resuspended in cold de-ionized water at 3.9% solids (2 L), and the pH of the mixture was adjusted to pH 8 using 5N NaOH. The solution was homogenized using 1 pass on a Microfluidics LM-10 homogenizer set at 22,000 psi or 1300 bar. The cell lysate was kept cool on ice. Chitosan was added to the cell lysate to a final concentration of 0.03% wt/v of cell lysate. The mixture was stirred for 1 hour on ice. The mixture was centrifuged at 3000×g, 5 minutes at 10° C. The clarified supernatant was concentrated 5× via ultrafiltration using a Pellicon 2 10 kDA filter cassette, washed with 2.5 volumes of cold water and concentrated 10× using a 10 kDA filter and lyophilized to a dry powder using a Columbia International Vacuum Freeze Dryer model FD50-B2A. A flow chart of this process of preparing protein isolate is shown in FIG. 1C.

### Chitosan and Acid Precipitation—Sample B060

Fermentation broth from continuous fermentation was collected and centrifuged. The supernatant was discarded. Collected biomass was resuspended in cold de-ionized water at 3.9% solids (2 L), and the pH of the mixture was adjusted to pH 8 using 5N NaOH. The solution was homogenized using 1 pass on a Microfluidics LM-10 homogenizer set at 22,000 psi or 1300 bar. The cell lysate was kept cool on ice. Chitosan was added to the cell lysate to a final concentration of 0.03% wt/v of cell lysate. The mixture was stirred for 1 hour on ice. The mixture was centrifuged at 3000×g, 5 minutes at 10° C. The clarified supernatant was transferred to a beaker and the pH was adjusted to pH 4.5 by the slow addition of 0.2 N sulfuric acid. The mixture was centrifuged at 3000×g, 5 minutes at 10° C. The clarified supernatant was transferred to a beaker and the pH was adjusted to pH 4.5 by the slow addition of 0.2 N sulfuric acid. The solution was mixed for 30 minutes and then centrifuged 3000×g, 5 min at 10° C. The supernatant was discarded and the protein pellet washed 1× with an equal volume of acidified water at pH 4.5, centrifuged at 3000×g, 5 min at 10° C. to pellet protein. The protein pellet was resuspended in water and the pH adjusted to pH 7 with sodium hydroxide. The final protein solution was lyophilized to a dry powder.

### High Speed Centrifugation and Acid Precipitation

Fermentation broth from continuous fermentation was collected, and a 30% suspension of biomass was prepared in 20 mM sodium phosphate buffer, pH 7.0. The re-suspended cells were homogenized using 1 pass through a Microfluidics LM-10 pneumatic cell disrupter set at 22,000 psi. The homogenate was kept cool on ice. The homogenate was centrifuged at 9000 rpm for 90 minutes at 4° C. in a Beckman J2-21 centrifuge with a JA-10 rotor to remove cell debris. The clarified supernatant was transferred to a beaker and the pH was adjusted to pH 4.5 by the slow addition of cold 0.2 N sulfuric acid. The solution was mixed for 30 minutes and then centrifuged 3000×g, 20 min at 10° C. The supernatant was discarded and the protein pellet washed 1× with an equal volume of acidified water at pH 4.5, centrifuged at 3000×g, 20 min at 10° C. to pellet protein. The protein pellet was resuspended in water and the pH adjusted to pH 7 with sodium hydroxide. The final protein solution was lyophilized to a dry powder.

### Example 5

### Nutrition Assessment of Methylococcus capsulatus Protein Isolate

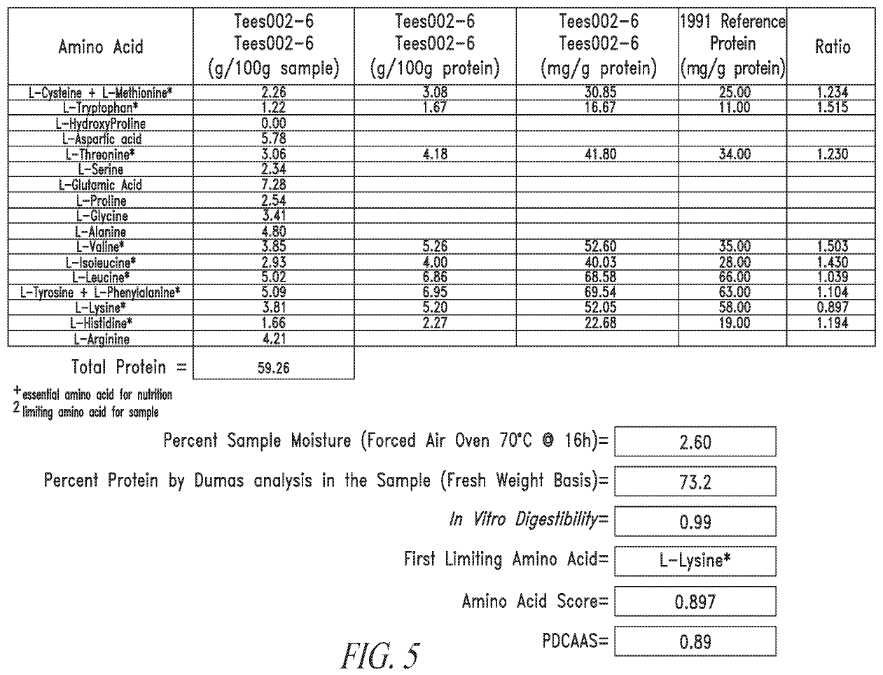

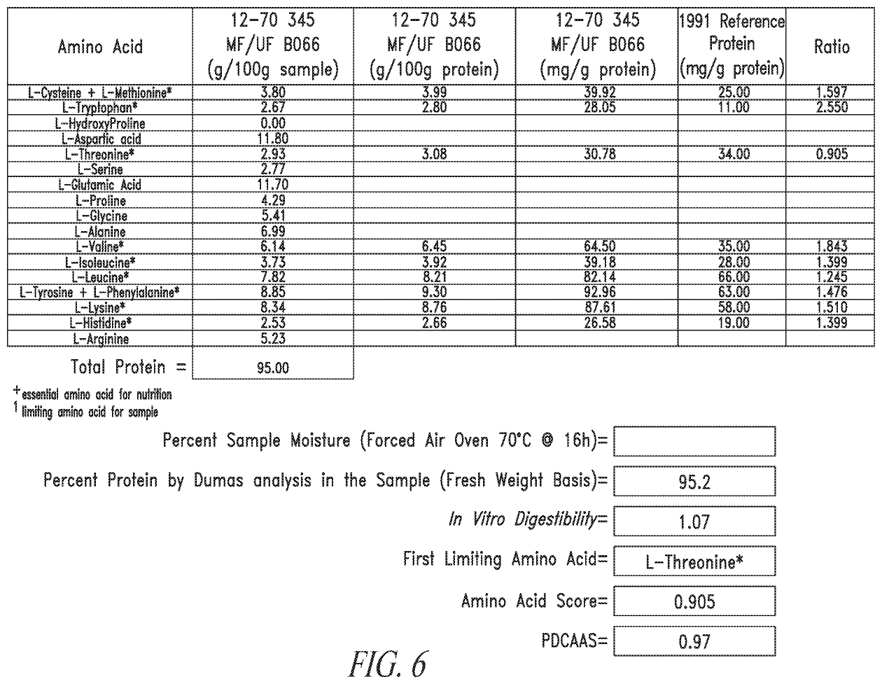

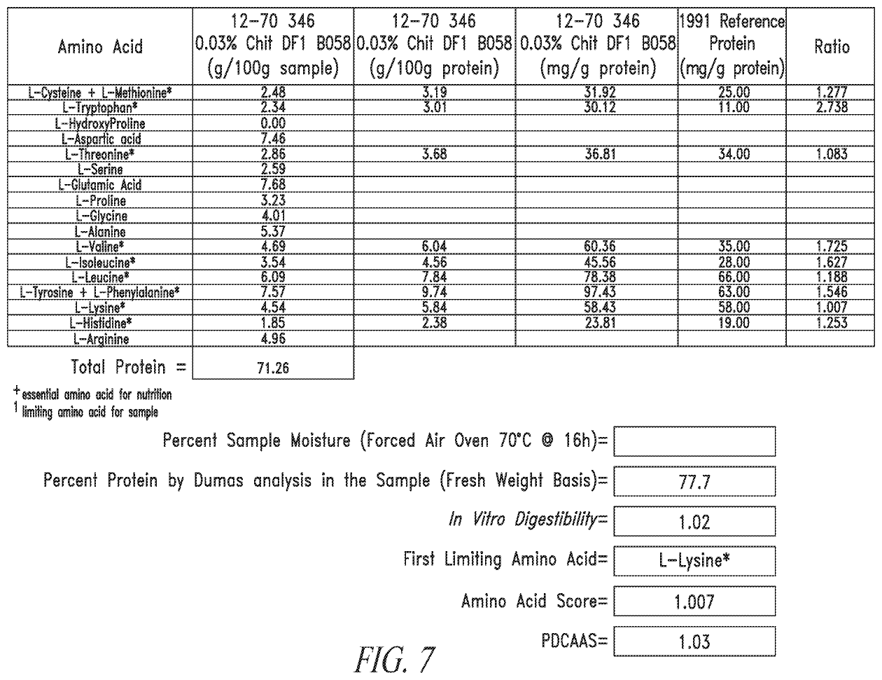

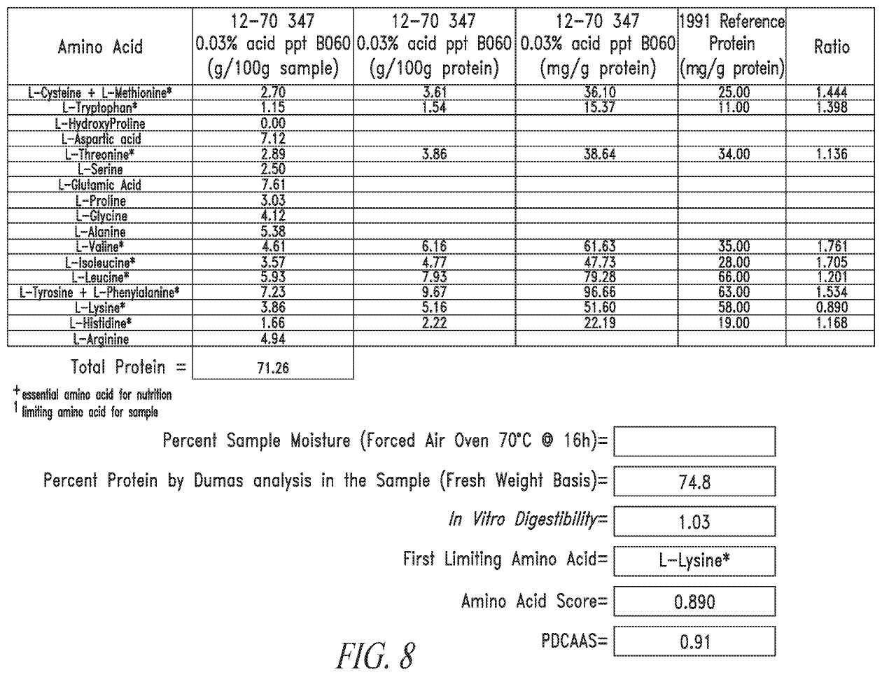

Protein digestibility-corrected amino acid score (PDCAAS) is a method of evaluating the quality of a protein based on both the presence of essential amino acids for human nutrition and their digestibility. Using the Protein Digestibility Assay Kit (Animal-Safe Accurate Protein Quality Score Method) (Megazyme), amino acid profile and PDCAAS were determined on Methylococcus capsulatus protein isolates prepared as described in Example 4 and whole cell products prepared as described in Example 16 or by a similar method. The Animal-Safe Accurate Protein Quality Score method is an in vitro enzyme digestion method that has a high correlation to the traditional in vivo rat digestion model. The results of a control sample and three exemplary protein isolates prepared according to Example 4 are shown in FIGS. 5-8: a control sample in FIG. 5; a sample of Methylococcus capsulatus protein isolate prepared by microfiltration/ultrafiltration method (B066) in FIG. 6, a sample of Methylococcus capsulatus protein isolate prepared using chitosan and ultrafiltration method (B058) in FIG. 7, and a sample of Methylococcus capsulatus protein isolate prepared using chitosan/acid precipitation method (B060) in FIG. 8. The control sample was prepared by concentrating, heat inactivating, and drying Methylococcus capsulatus biomass. Methylococcus capsulatus protein isolates had PDCAAS scores of at least 0.90 and higher than most plant-based proteins (FIG. 9).

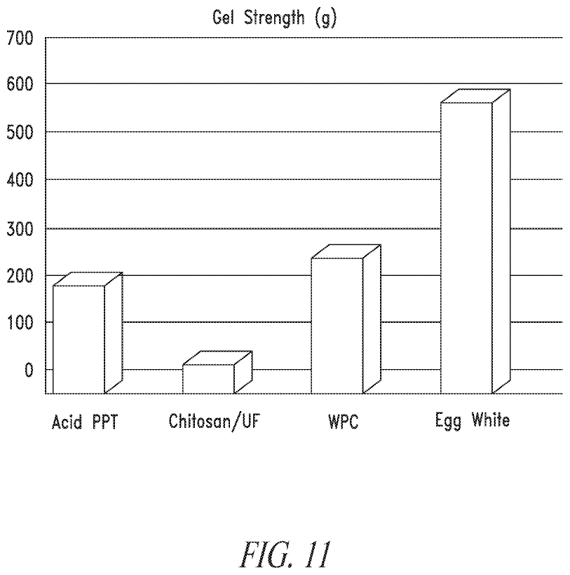

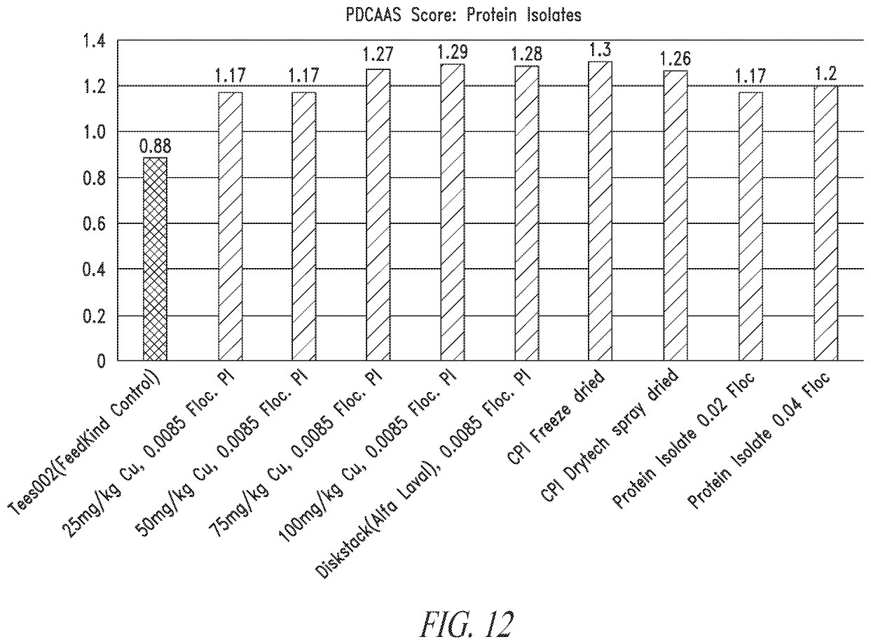

The PDCAAS values of several additional protein isolates are shown in FIG. 12. The control as described in FIG. 5 is included as a control in FIG. 12. The various protein isolates were prepared as follows: the copper concentration used in the fermentation was 125 mg/kg (unless otherwise specified), the biomass produced by fermentation was homogenized, mixed with chitosan flocculent (amount of chitosan added varying, expressed in g of chitosan/g DCW (dry cell weight) of biomass), clarified by centrifugation, concentrated by ultrafiltration and dried (freeze dried otherwise specified).

More specifically, the various protein isolates were prepared as follows:
(a) The concentration of copper in the fermentation broth was varied from 25 mg/kg Cu to 100 mg/kg Cu, lab scale floor centrifuge and 0.0085 g of chitosan/g DCW biomass were used to generate the following protein isolate samples
25 mg/kg Cu, 0.0085 Floc. PI
50 mg/kg Cu, 0.0085 Floc. PI
75 mg/kg Cu, 0.0085 Floc. PI
100 mg/kg Cu, 0.0085 Floc. PI
(b) Lab scale disk stack centrifuge with amount of chitosan varying from 0.0085 to 0.04 g of chitosan/g DCW biomass was used to generate the following protein isolate samples
Disk stack (Alfa Laval), 0.0085 Floc. PI
Protein Isolate 0.02 Floc
Protein Isolate 0.04 Floc
(c) 0.0085 of chitosan/g DCW biomass was used in a pilot scale production to generate the following protein isolate samples.
CPI Freeze dried—protein isolate freeze dried
CPI Drytech spray dried—protein isolate spray dried

The PDCAAS values of three whole cell products were also measured. Two of the whole cell products were prepared according to Example 16, and the third whole cell product was produced with an additional homogenization step after the heat treatment. Their PDCAAS values were 0.98, 1.02, and 1.02.

The results shown that the average PDCAAS values were 0.89 for the control sample, 1.01 for the three whole cell products, and 1.23 for the protein isolates analyzed in FIG. 12.

The protein isolates whose PDCAAS values are shown in FIG. 12 were also subject to Dumas analysis to determine their crude protein percentages by weight. The results are shown in FIG. 18.

The control sample and three whole cell products described above were also subject to Dumas analysis to determine their crude protein amounts. The results are 73.2% (for the control sample), 74.1%, 70.6% and 70.0%.

### Example 6

### Gelation Properties of Methylococcus capsulatus Protein Isolate

Gelation property of Methylococcus capsulatus protein isolates prepared as described in Example 4 was measured using a Brookfield CT3 Texture Analyzer. 34 g of distilled water was added to a 100 ml beaker (with a stir bar). The beaker was placed on a stirrer, and 6 g of protein powder was gradually added to the beaker. pH of the protein solution was adjusted to 7.0 using 1N NaOH or HCl. The protein solution was stirred for one hour. The protein solution settled for 30 minutes to allow foam to dissipate. The protein solution was poured into a casing (34 mm flat), tied, and sealed with cable ties. The encased protein solution was heated in a 75° C. water bath for 60 minutes. The encased protein was cooled in running tap water for one hour. The encased protein was stored in the refrigerator overnight and then placed in running tap water for one hour. The casing was carefully removed from the gelled protein, and the maximum stress force of the gelled proteins was measured using the Brookfield CT3 Texture Analyzer with a cylinder probe 12.5 mm. As shown in FIG. 11, whey protein concentrate (WPC) had a gel strength of about 280 g. The initial strength was low to start with and then increased with compression to 280 g. The Methylococcus capsulatus protein isolate gel (“Chitosan/UF”) prepared from the chitosan and ultrafiltration method described in Example 4 was fragile when the casing was cut and not elastic. The compressing strength increased quickly and then crashed quickly. The sample comprising Methylococcus capsulatus protein isolate prepared by high speed centrifugation and acid precipitation as described in Example 4 was elastic and had good gel strength (FIGS. 10-11).

### Example 7

### Solubility of Methylococcus capsulatus Protein Isolate

Solubility of Methylococcus capsulatus protein isolates prepared by chitosan and ultrafiltration process (“UF”) or high speed centrifugation and acid precipitation process as described in Example 4 was measured. For the “UF” M. capsulatus protein isolates, biomass was preheated at 65° C. for 10 seconds prior to isolating the protein from the biomass, biomass was preheated at 65° C. for 5 seconds prior to isolating the protein from the biomass, or the biomass was not preheated prior to isolating the protein from the biomass. Two 2.0% protein solutions were prepared for each sample in 100 ml beakers (0.5 g protein isolate in 24 g water). pH of one of the solutions was adjusted to 7.0 with NaOH, and the pH of the other solution was adjusted to 4.3 with HCl. The weight of both solutions was balanced to 25 g with water. The solutions were stirred for one hour at room temperature, and 1.5 ml protein solution was centrifuged at 7,000 rpm (4000×g) in Eppendorf 5414C microcentrifuge for 10 minutes. The protein contents of the 2% solution and the supernatant were measured using a Dumas Protein Analyzer, which makes a quantitative determination of nitrogen content by combustion and analysis of the evolved N₂ gas. % solubility was calculated by dividing the protein content of the supernatant by the protein in the initial sample (×100%) (FIGS. 17A-17B). Acid precipitated Methylococcus capsulatus protein isolates exhibited nearly 100% solubility at pH 7.0 and 0% solubility at pH 4.3.

Protein content of the Methylococcus capsulatus protein isolates were also measured for protein solutions made from M. capsulatus isolates made by chitosan and ultrafiltration process with pre-heating of biomass at 65° C. for 5 seconds prior to processing or high speed centrifugation and acid precipitation (FIGS. 18A-18B). 2.0% protein solutions were prepared for each sample using 1 g protein isolate in 49 g water. 24 g trichloroacetic acid (TCA) and 76 g water were used to prepare a 24% TCA solution. Equal volumes of the protein solution and 24% TCA (20 mls+20 mls) were mixed together and then centrifuged at 4000×g for 10 minutes (7000 rpm by Eppendorf 5414C microcentrifuge). The nitrogen (protein) content of the 2% protein solution and the nitrogen (protein) content of the supernatant were determined using the Dumas method. Total (crude) protein=(protein content of 2% solution)/0.02. % non-protein nitrogen (NPN)=2×(nitrogen content of the supernatant)/0.02×100. % True protein=Total Protein−% NPN.

### Example 8

### Emulsion Test of Methylococcus capsulatus Protein Isolate

Emulsion test was also performed on Methylococcus capsulatus protein isolates. 2% protein isolate, 82% water (3 g protein isolate, 24 g vegetable oil and 123 g water) and optionally stabilizers (0.05-0.25% sodium hexametaphosphate, 0.1-0.5% DATEM) were mixed with a hand held blender and optionally heated to 50° C. and blended with 16% vegetable oil using a microfluidizer at 2500 psi. Previous testing revealed that Methylococcus capsulatus protein isolates were heat sensitive and did not make good emulsions. Upon removal of the preheating step (“NH”) or addition of an emulsifying agent (e.g., phosphates+DATEM), the Methylococcus capsulatus protein isolates made stable emulsions (FIGS. 13-14B).

### Example 9

### Foaming Test of Methylococcus capsulatus Protein Isolate

4% protein solutions were prepared by adding 2 g Methylococcus capsulatus protein isolate powder produced using the high speed centrifugation and acid precipitation method as described in Example 4 to 48 g water, adjusting pH to 7.0. 20 ml of 4% Methylococcus capsulatus protein solution was added to a 100 ml beaker. The temperature of the liquid was recorded with a digital thermometer. The sample was whisked for 2 minutes using an electric whisk or milk frother by positioning the whisk at the liquid/air interface. The height of any foam produced was recorded, using a gas pycnometer (11.5 ml capacity, ⅛″ hole in lid, Cole-Parmer Part #EW-38001-00). The foam volume was calculated using the following formula:

Overrun (%)=(Wt. of solution−Wt. of foam)/(Wt. of foam)×100

To measure foam stability, 15 mls of 4% Methylococcus capsulatus protein solution was whipped in a 50 ml graduated cylinder for 2 minutes. The height of any foam produced was recorded by marking on the outside of the conical tube. The sample was allowed to stand idle for 30 minutes, either at room temperature of at 20° C., depending on the initial condition of the sample. The sample was then monitored for separation of the foam into a foam and liquid phase to determine foam stability. The height of any remaining foam was measured using the marks on the outside of the conical tube. Foam stability was calculated using the following formula:

Foam Stability (%)=(Residual foam volume)/(Total foam volume)×100

As shown in Table 3 and FIGS. 15A-15C, the sample containing Methylococcus capsulatus Bath protein isolate exhibited good foaming, despite the presence of anti-foaming agent during the fermentation process. Egg white was used as a control sample.

TABLE 3
Foam overrun

| Sample | Overrun |
|---|---|
| Egg White | 279% |
| Methylococcus capsulatus Bath protein | 584% |
| isolate (acid precipitated) | |

### Example 10

### Meringue Cookies Comprising Methylococcus capsulatus Protein Isolate

Meringue cookies were prepared according to the compositions provided in Table 4. A protein solution (egg white or Methylococcus capsulatus Protein Isolate) was prepared with water, mixed with cream of tartar, and beaten with a portable electric mixer on low speed for 1 minute or until soft peaks form. Sugar was gradually added, beating on high speed about 5 minutes or until mixture formed stiff, glossy peaks and sugar dissolved (rub a small amount of batter between two fingers, it should feel completely smooth). Foamy batter was deposited onto a baking sheet to make ˜10 cookies and baked approximately 1 hour at 200° F. until moisture was 2%.

TABLE 4
Meringue cookie compositions

| | | | | | Methylococcus | |
|---|---|---|---|---|---|---|
| | 3 g | | 5 g | | capsulatus Bath | |
| | protein/oz. | | protein/oz. | | Protein Isolate | |
| Ingredient | Composition | | Composition | | Composition | |
| Water | 20 | g | 16 | g | 18 | g |
| Protein | 2.8 | g | 6 | g | 5 | g |
| Sugar | 17.2 | g | 17.6 | g | 15 | g |
| Cream of Tartar | 0.4 | g | 0.4 | g | 0.4 | g |

Meringue batter made using Methylococcus capsulatus Bath protein isolate formed peaks (FIG. 16A). The Methylococcus capsulatus Bath protein isolate imparted a mild flavor with some pink color in the meringue cookies (FIG. 16B).

### Example 11

### Meat Substitute Comprising Methylococcus capsulatus Protein Isolate

Methylococcus capsulatus Bath protein isolate possesses heme-containing proteins, which confer meat-substitute food compositions fresh meat color that darkens during cooking, a slightly metallic taste, and the smell of meat. The good gelation property of Methylococcus capsulatus Bath protein isolate also provides improved texture. Methylococcus capsulatus Bath protein isolate also possesses a high protein nutrition profile.

An exemplary meat substitute composition is provided in Table 5. Dehydrated textured soy protein (Dupont, Response 4310 IP Textured Soy Protein Concentrate, minimum 6900 protein) (FIG. 19A) was soaked in water to rehydrate before use. The dry weight of the textured soy protein is about 22.2%. Methylococcus capsulatus protein isolate prepared using the chitosan and ultrafiltration method described in Example 4, flour, oil, and other spices were added to the hydrated textured soy protein and mixed together. The mixture was shaped into a patty and frozen prior to cooking (FIG. 19B). The patty had fresh meat color and turned darker and brown when cooked (FIG. 19C). The cooked meat-substitute patty made from Methylococcus capsulatus protein isolate exhibited improved texture, good gelation and water binding properties (FIG. 19D)

TABLE 5
Meat-substitute composition

| | |
|---|---|
| Flour | 9% |
| Methylococcus capsulatus Bath protein | 15.7% |
| isolate (chitosan and ultrafiltration) | |
| Coconut oil | 5.4% |
| Salt | 0.9% |
| Garlic powder | 0.7% |
| Onion powder | 0.7% |
| Hydrated Textured Soy Protein | 67.6% |
| | (15% dry textured soy |
| | protein + 52.6% water) |
| Protein in Methylococcus capsulatus | 12.1% |
| Bath protein isolate | |
| Protein from Dry Textured Soy | 10.4% |
| Total Protein in meat-substitute composition | 22.5% |

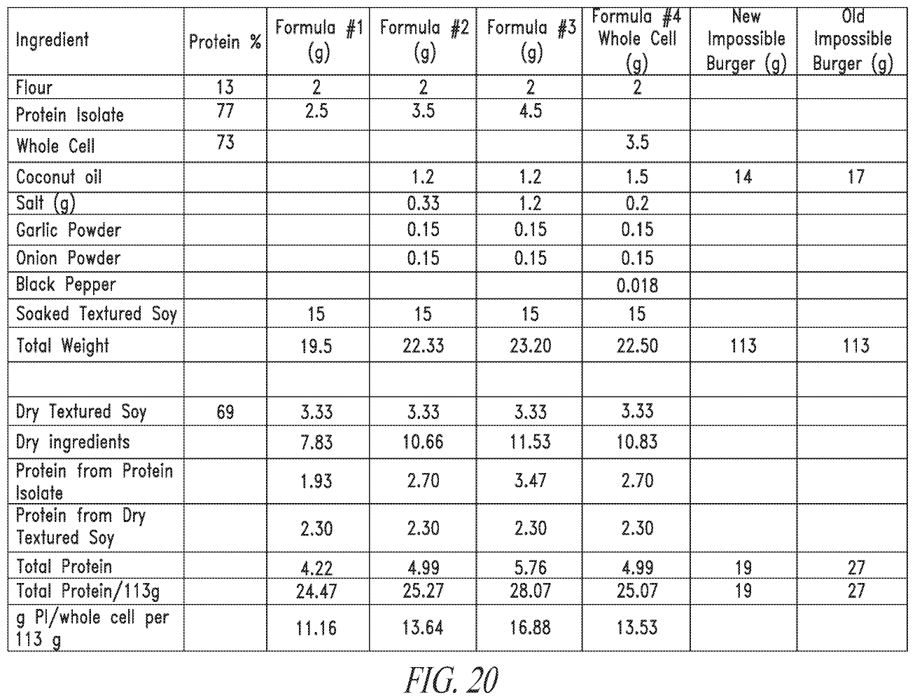

Additional meat-substitute patty compositions for Methylococcus capsulatus Bath protein isolate (chitosan and ultrafiltration) are also shown in FIG. 20.

### Example 12

### Non-Dairy Ice Cream Comprising Methylococcus capsulatus Bath Protein Isolate

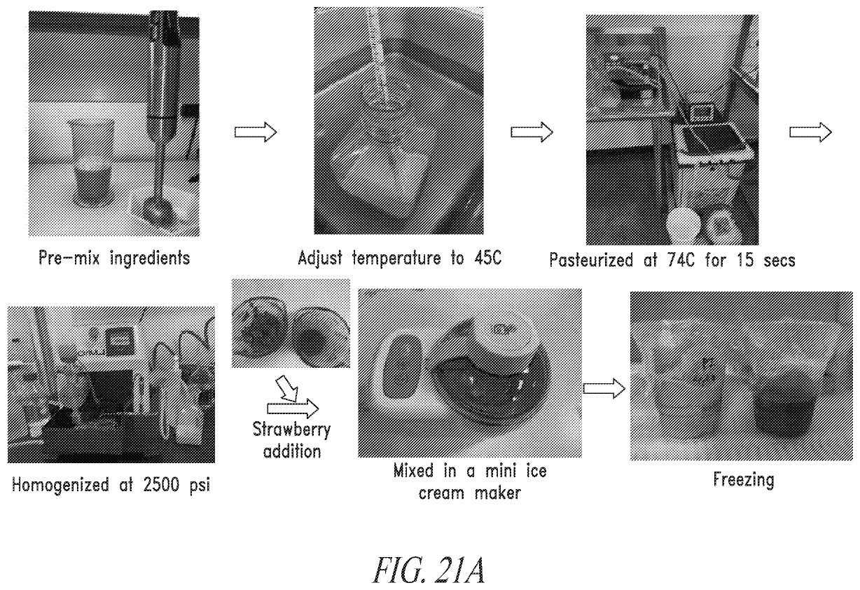

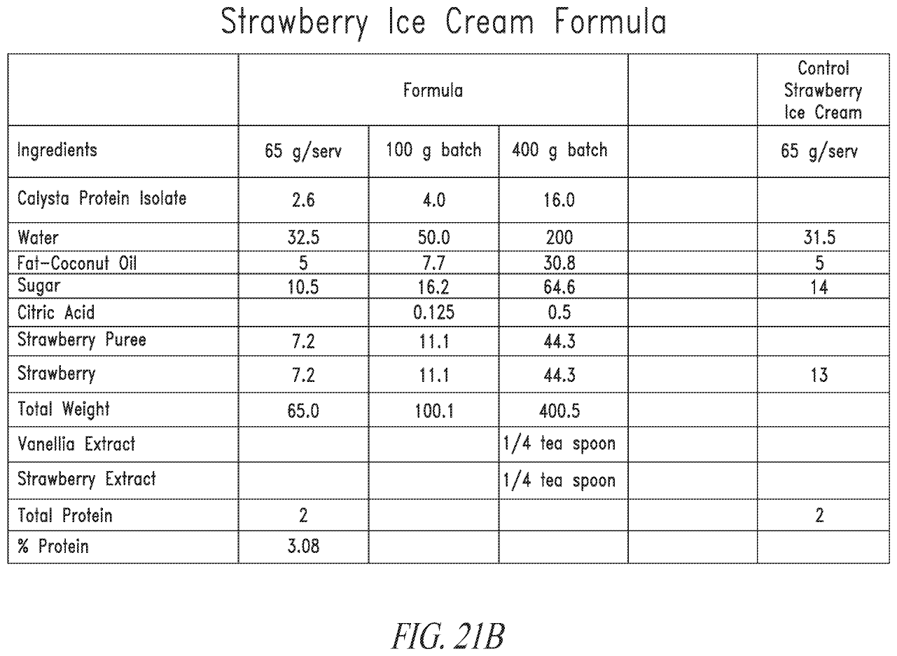

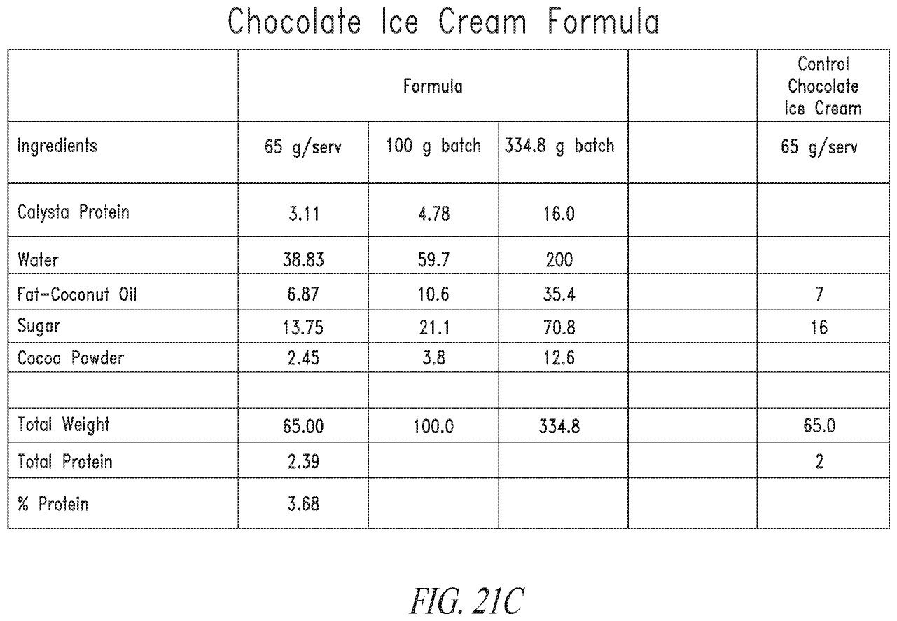

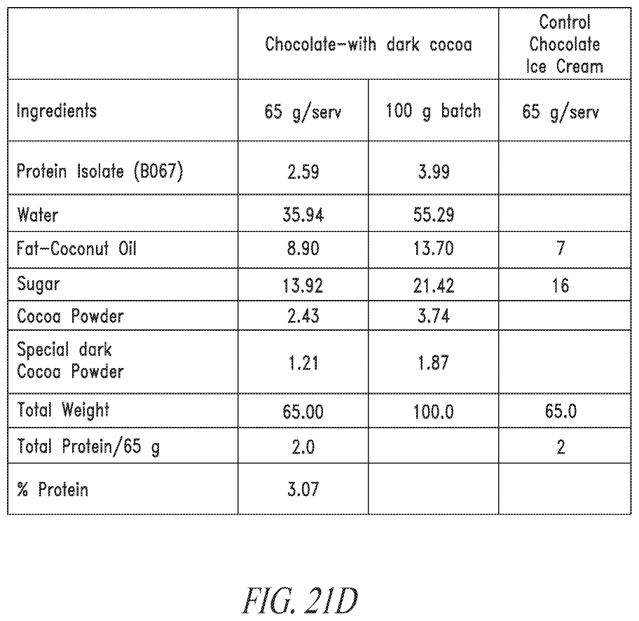

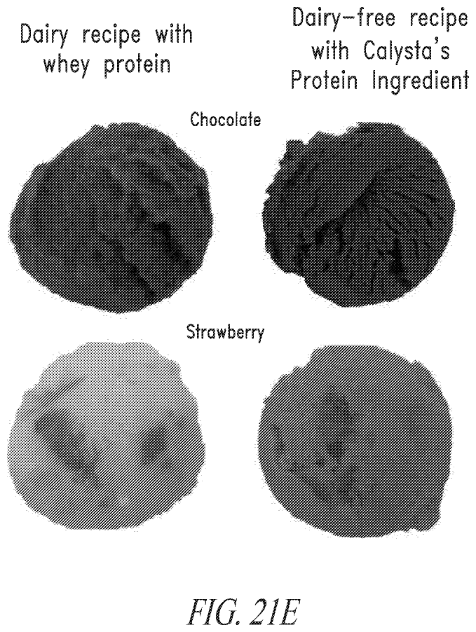

Strawberry and chocolate ice cream comprising Methylococcus capsulatus protein isolate (prepared by chitosan and ultrafiltration method in Example 4) was prepared according to the process and compositions shown in FIGS. 21A-21D. The chocolate ice cream comprising Methylococcus capsulatus protein isolate had a smoother texture and good mouth-feel compared to a control chocolate ice cream made with whey protein concentrate.

### Example 13

### Snacks Fortified with Methylococcus capsulatus Protein Isolate

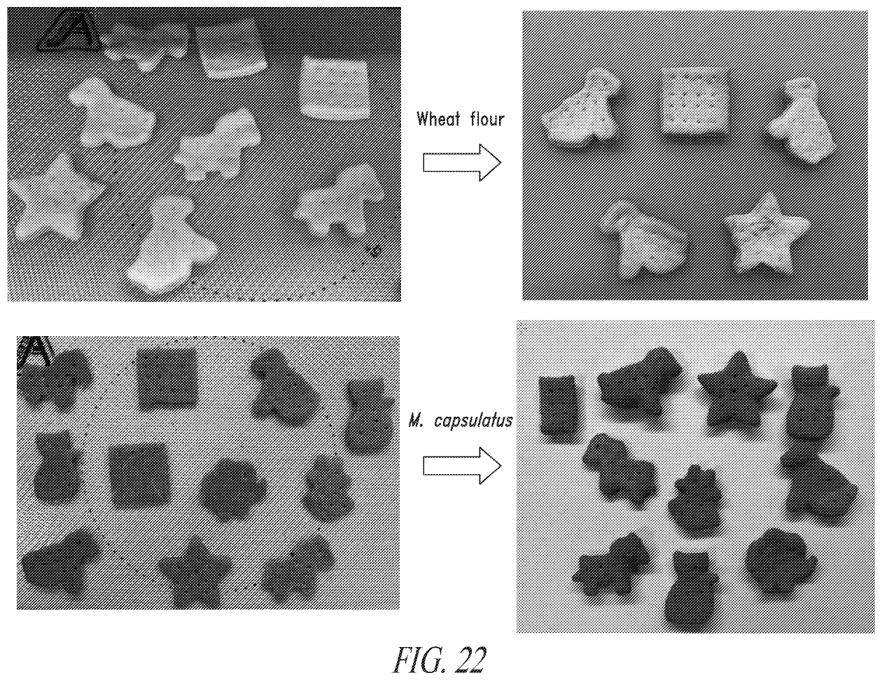

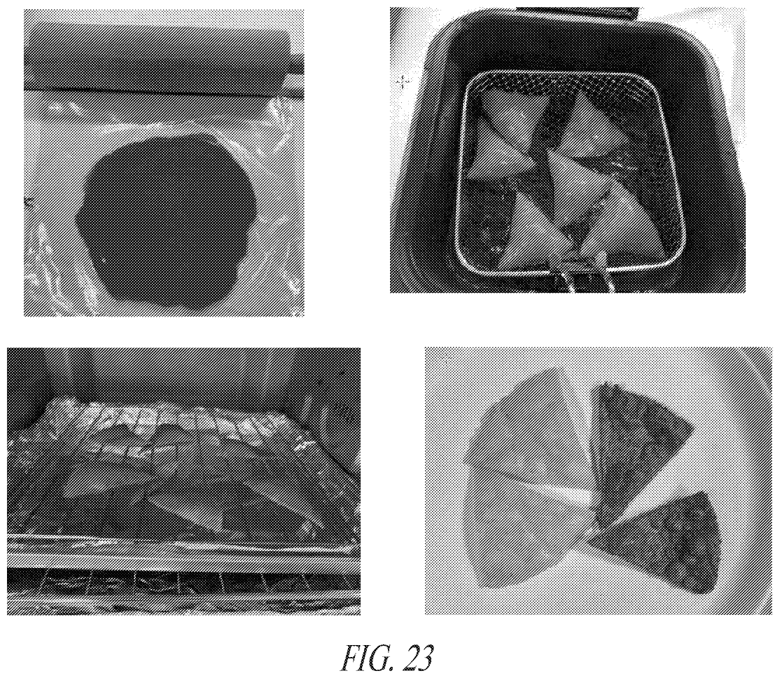

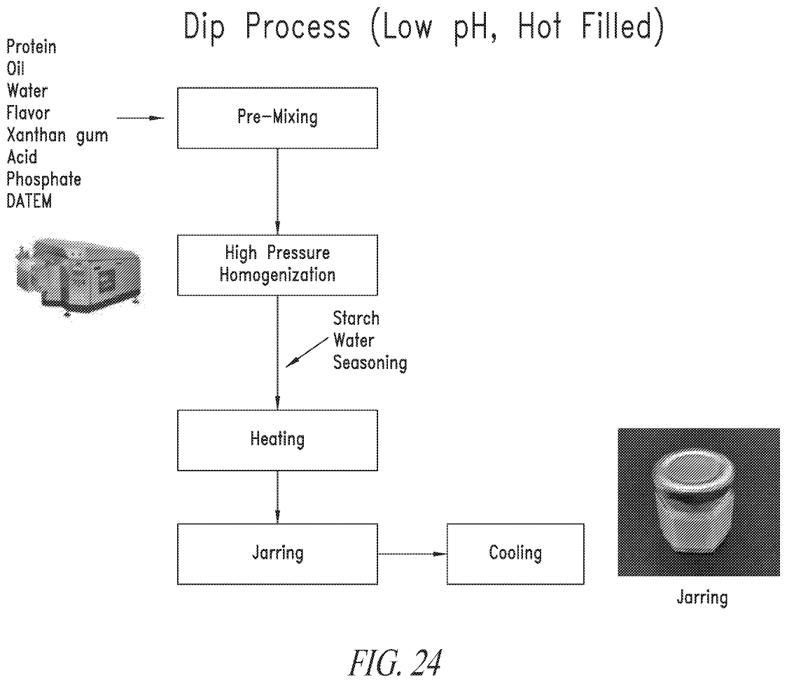

Crackers were made with either wheat flour or fortified with Methylococcus capsulatus protein isolate (prepared using chitosan and ultrafiltration method described in Example 4) according to the composition shown in Table 6. The cracker dough was rolled flat, approximately 12 cracker shapes were cut from the dough (˜2 g/cracker), and the crackers were baked at 350F for −25 minutes or until brown. As shown in FIG. 22, the Methylococcus capsulatus Bath protein isolate imparted the cracker dough with a red color, which turned brown during baking.

TABLE 6
Cracker composition

| | Methylococcus capsulatus | | Wheat Flour | |
|---|---|---|---|---|
| | Bath protein isolate | | Control | |
| Ingredients | Composition | | Composition | |
| Wheat Flour | 16 | g | 19.75 | g |
| Methylococcus capsulatus | 3.75 g (16.7%) | | 0 | g |
| Bath protein isolate | | | | |
| Sodium bicarbonate | 0.15 | g | 0.15 | g |
| Sugar | 1.35 | g | 1.35 | g |
| Salt | 0.13 | g | 0.13 | g |
| Dimodan (emulsifier) | 0.24 | g | 0.24 | g |
| Calcium carbonate | 0 | g | 0 | g |
| Ammonium bicarbonate | 0.08 | g | 0.08 | g |
| Vegetable oil | 1.1 | g | 1.1 | g |
| Dry ingredients | 23.36 | g | 23.36 | g |
| Protein % (>22.32%) | 22.48 | | 11.53 | |
| Water | 9.5 | g | 9.5 | g |

Chips fortified with Methylococcus capsulatus Bath protein isolate (prepared by chitosan and ultrafiltration method in Example 4) were made according to the composition in Table 7 below. Aquamin™ mineral supplement, which contains calcium carbonate, was added at 1.52 wt to improve texture and color (see, FIG. 23). The dough was rolled flat. Chips were cut from the flattened dough, and baked at 250° F. before frying in 350° F. oil. The resulting chips contain 5 g protein/serving.

TABLE 7
Chip composition

| Ingredient | Weight (g) |
|---|---|
| Masa | 22.9 |
| Methylococcus capsulatus Bath Protein Isolate | 7.1 |
| Aquamin mineral supplement | 0.45 |
| Water | 26.5 |

### Example 14

### Effect of Methane Source and Purity on Stable Carbon Isotope Distribution

To examine methanotroph growth on methane containing natural gas components, a series of 0.5-liter serum bottles containing 100 mL defined media MMS1.0 were inoculated with Methylosinus trichosporium OB3b or Methylococcus capsulatus Bath from a serum bottle batch culture (5% v/v) grown in the same media supplied with a 1:1 (v/v) mixture of methane and air. The composition of medium MMS1.0 was as follows: 0.8 mM MgSO₄*7H₂O, 30 mM NaNO₃, 0.14 mM CaCl₂), 1.2 mM NaHCO₃, 2.35 mM KH₂PO₄, 3.4 mM K₂HPO₄, 20.7 μM Na₂MoO₄*2H₂O, 6 μM CuSO₄*5H₂O, 10 μM Fe^{III}—Na-EDTA, and 1 mL per liter of a trace metals solution (containing, per L: 500 mg FeSO4*7H₂O, 400 mg ZnSO₄*7H₂O, 20 mg MnCl₂*7H₂O, 50 mg CoCl₂*6H₂O, 10 mg NiCl₂*6H₂O, 15 mg H₃BO₃, 250 mg EDTA). Phosphate, bicarbonate, and Fe^{III}—Na-EDTA were added after media was autoclaved and cooled. The final pH of the media was 7.0±0.1.

The inoculated bottles were sealed with rubber sleeve stoppers and injected with 60 mL methane gas added via syringe through sterile 0.45 μm filter and sterile 27G needles. Duplicate cultures were each injected with 60 mL volumes of (A) methane of 99% purity (grade 2.0, Praxair through Alliance Gas, San Carlos, CA), (B) methane of 70% purity representing a natural gas standard (Sigma-Aldrich; also containing 9% ethane, 6% propane, 3% methylpropane, 3% butane, and other minor hydrocarbon components), (C) methane of 85% purity delivered as a 1:1 mixture of methane sources A and B; and (D) >93% methane (grade 1.3, Specialty Chemical Products, South Houston, TX; in-house analysis showed composition >99% methane). The cultures were incubated at 30° C. (M. trichosporium strain OB3b) or 42° C. (M. capsulatus Bath) with rotary shaking at 250 rpm and growth was measured at approximately 12 hour intervals by withdrawing 1 mL samples to determine OD₆₀₀. At these times, the bottles were vented and headspace replaced with 60 mL of the respective methane source (A, B, C, or D) and 60 mL of concentrated oxygen (at least 85% purity). At about 24 hour intervals, 5 mL samples were removed, cells recovered by centrifugation (8,000 rpm, 10 minutes), and then stored at −80° C. before analysis. Analysis of carbon and nitrogen content (% dry weight), and carbon (¹³C) and nitrogen (¹⁵N) stable isotope ratios, for methanotrophic biomass derived from M. trichosporium strain OB3b and M. capsulatus Bath were carried out. Table 8 shows the results of stable carbon isotope analysis for biomass samples from M. capsulatus Bath grown on methane having different levels of purity and in various batches of bottle cultures.

TABLE 8
Stable Carbon Isotope Distribution of M. capsulatus Bath
Grown on Different Methane Sources having Different Purity

| Methane* | Batch No. | Time (h)† | OD₆₀₀ | DCW (g/L) | δ¹³C Cells |
|---|---|---|---|---|---|
| A | 62C | 22 | 1.02 | 0.36 | −40.3 |
| | | 56 | 2.01 | 0.71 | −41.7 |
| | | 73 | 2.31 | 0.82 | −42.5 |
| | 62D | 22 | 1.14 | 0.40 | −39.3 |
| | | 56 | 2.07 | 0.73 | −41.6 |
| | | 73 | 2.39 | 0.85 | −42.0 |
| B | 62E | 22 | 0.47 | 0.17 | −44.7 |
| | | 56 | 0.49 | 0.17 | −45.4 |
| | | 73 | 0.29 | 0.10 | −45.4 |
| | 62F | 22 | 0.62 | 0.22 | −42.3 |
| | | 56 | 0.63 | 0.22 | −43.6 |
| | | 73 | 0.30 | 0.11 | −43.7 |
| C | 62G | 22 | 0.70 | 0.25 | −40.7 |
| | | 56 | 1.14 | 0.40 | −44.8 |
| | | 73 | 1.36 | 0.48 | −45.8 |
| | 62H | 22 | 0.62 | 0.22 | −40.9 |
| | | 56 | 1.03 | 0.37 | −44.7 |
| | | 73 | 1.23 | 0.44 | −45.9 |

*Methane purity: A: 99% methane, grade 2.0 (min. 99%); B: 70% methane, natural gas standard (contains 9% ethane, 6% propane, 3% methylpropane, 3% butane); C: 85% methane (1:1 mix of A and B methane)
†Time = bottle culture time in hours

The average δ¹³C for M. capsulatus Bath grown on one source of methane (A, 99%) was −41.2±1.2, while the average δ¹³C for M. capsulatus Bath grown on a different source of methane (B, 70%) was −44.2±1.2. When methane sources A and B were mixed, an intermediate average δ¹³C of −43.8±2.4 was observed. These data show that the δ¹³C of cell material grown on methane sources A and B are significantly different from each other due to the differences in the δ¹³C of the input methane. However, cells grown on a mixture of the two gasses preferentially utilize ¹²C and, therefore, show a trend to more negative δ¹³C values.

A similar experiment was performed to examine whether two different methanotrophs, Methylococcus capsulatus Bath and Methylosinus trichosporium OB3b, grown on different methane sources and in various batches of bottle cultures showed a difference in δ¹³C distribution (see Table 9).

TABLE 9
Stable Carbon Isotope Distribution of Different Methanotrophs Grown
on Different Methane Sources of Different Purity

| | | Batch | Time | | DCW | δ¹³C |
|---|---|---|---|---|---|---|
| Strain | Methane* | No. | (h)† | OD₆₀₀ | (g/L) | Cells |
| Mc Bath | A | 62I | 18 | 0.494 | 0.18 | −54.3 |
| | | | 40 | 2.33 | 0.83 | −42.1 |
| | | | 48 | 3.08 | 1.09 | −37.1 |
| Mc Bath | D | 62J | 18 | 0.592 | 0.21 | −38.3 |
| | | | 40 | 1.93 | 0.69 | −37.8 |
| | | | 48 | 2.5 | 0.89 | −37.8 |
| Mc Bath | D | 62K | 18 | 0.564 | 0.20 | −38.6 |
| | | | 40 | 1.53 | 0.54 | −37.5 |
| | | | 48 | 2.19 | 0.78 | −37.6 |
| Mt OB3b | A | 68D | 118 | 0.422 | 0.24 | −50.2 |
| | | | 137 | 0.99 | 0.55 | −47.7 |
| | | | 162 | 1.43 | 0.80 | −45.9 |
| Mt OB3b | A | 68E | 118 | 0.474 | 0.26 | −49.9 |
| | | | 137 | 1.065 | 0.59 | −47.6 |
| | | | 162 | 1.51 | 0.84 | −45.2 |
| Mt OB3b | D | 68F | 118 | 0.534 | 0.30 | −45.6 |
| | | | 137 | 1.119 | 0.62 | −38.7 |
| | | | 162 | 1.63 | 0.91 | −36.4 |
| Mt OB3b | D | 68G | 118 | 0.544 | 0.30 | −44.8 |
| | | | 137 | 1.131 | 0.63 | −39.1 |
| | | | 162 | 1.6 | 0.89 | −34.2 |

*Methane sources and purity: A: 99% methane (grade 2.0); D: >93% methane (grade 1.3)
†Time = bottle culture time in hours

The average δ¹³C for M. capsulatus grown on a first methane source (A) was −44.5±8.8, while the average δ¹³C for M. trichosporium was −47.8±2.0 grown on the same methane source. The average δ¹³C for M. capsulatus grown on the second methane source (B) was −37.9±0.4, while the average δ¹³C for M. trichosporium was −39.8±4.5. These data show that the δ¹³C of cell material grown on a methane source is highly similar to the δ¹³C of cell material from a different strain grown on the same source of methane. Thus, the observed δ¹³C of cell material appears to be primarily dependent on the composition of the input gas rather than a property of a particular bacterial strain being studied.

### Example 15

### Heme Concentration in Methylococcus capsulatus Protein Isolate

The concentration of heme was measured based on the conversion of heme to the fluorescent porphyrin derivative by removal of the heme iron under acidic conditions as described in Sassa, Journal of Experimental medicine 143: 305-315, 1976. Heme iron was calculated using the relationship of 1 mole of heme iron/mole of heme. Three batches of M. capsulatus protein isolate prepared using chitosan flocculation followed by ultrafiltration as described in Example 4 and their heme and heme iron concentrations as shown in Table 10.

TABLE 10
Heme and hemo iron concentrations of M. capsulatus protein isolate

| Batch | mg heme/g protein | mg heme iron/g protein |
|---|---|---|
| B001 | 0.450 | 0.0408 |
| B110 | 0.370 | 0.0335 |
| SK173 | 0.680 | 0.0616 |

### Example 16

### Preparing Methylococcus capsulatus Whole Cell Products

Centrifuged bacterial biomass obtained from continuous fermentation of M. capsulatus was re-suspended in cold de-ionized water to 8% or 13-14% total solids. The slurry was mixed until homogeneous and was pH adjusted to 7 with 5N NaOH. Next, the entire volume of bacterial biomass was pumped through a 5 m heating coil submerged in a 90° C. water bath using a Watson Marlow 505U peristaltic pump. The pump rate was set at 75 RPM and calculated to achieve a ten second residence time for the fresh biomass at 90° C. Heated biomass was collected in a vessel that was being held at 60° C. with moderate stirring. Once the entire volume had gone through into the vessel, it was held for an additional 30 minutes, then cooled below to <20° C. Biomass was now dried or concentrated via ultrafiltration (UF). 8% total solid whole cell product was concentrated 2-3× via using a Pellicon 2 10 kDa filter cassette ultrafiltration. The concentrated whole cell was freeze dried using a Columbia International Vacuum Freeze Dryer model FD50-B2A. 13-14% total solid whole cell product was directly freeze dried in a HarvestRight HR9000-AL freeze dryer. The final dried whole cell products were characterized for % crude protein using the Dumas method and the nucleic acid content was determined using a Lucigen Masterpure Complete DNA & RNA Purification Kit MC85200. The process is illustrated in FIG. 27.

Whole cell product was shown to have no change in % crude protein compared to no heat shock biomass grown with normal continuous fermentation conditions, at 72-74%. The nucleic acid content of 8% starting solids was 2-3% total weight of the whole cell product. The nucleic acid content of 13-14% starting solids was higher, at 3.6-4.7% total weight of the whole cell product.

### Example 17

### Meat Substitute Comprising Methylococcus capsulatus Whole Cell Products

Whole cell products from Methylococcus capsulatus manufactured with or without homogenization were formulated in hamburger patties. The colors of both hamburger patties were lighter compared to the color of the hamburger formulated with Methylococcus capsulatus protein isolates (e.g., protein isolates produced by chitosan flocculation followed by ultrafiltration).

During frying/grilling, the color of both patties with whole cell products turned darker and brown from pinkish color (see FIG. 19E). Their textures were softener and their color were lighter than the patties with protein isolated prepared from chitosan flocculation process. The patty with homogenized whole cell product was darker than the one without homogenization.

An exemplary recipe for making hamburger patties using whole cell products are in FIG. 20.

### Example 18

### Marking Yogurt Comprising Methylococcus capsulatus Protein Isolates or Whole Cell Products

The protein isolates and whole cell products prepared from Methylococcus capsulatus biomass can be formulated to non-dairy or non-animal yogurt. They can be used in yogurt formula to replace animal milk to develop non-dairy, non-animal, plant-based yogurts or Greek yogurts.

Specifically, the protein isolates can function as an emulsifier to produce protein stabilized oil-in water. Both the protein isolates and whole cell products are good organic substrates for the lactic bacteria culture.

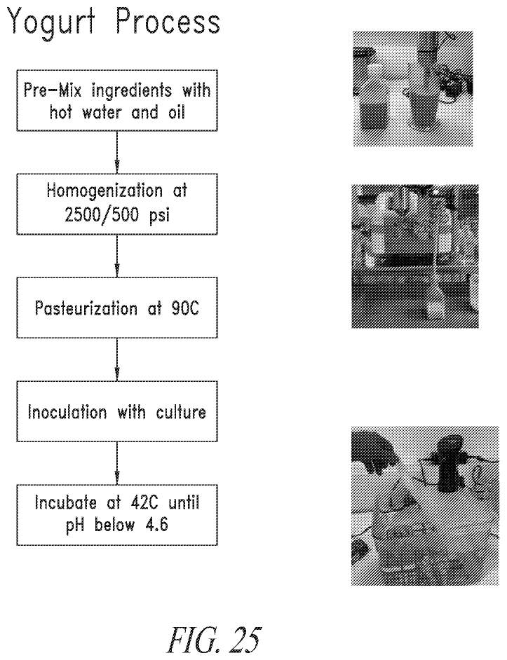

M. capsulatus protein isolate or whole cell product was hydrated with water and blended with oil, sugar (glucose, sucrose, fructose), minerals (calcium carbonate, sodium phosphates) and other stabilizers to mimic the composition of milk. After homogenization and pasteurization, the lactic bacteria culture or a proprietary plant-based yogurt culture (YoFlec YF-L02 DA from CHR Hansen) was inoculated to produce yogurts or Greek yogurts without using dairy milk. A scheme of an exemplary process of making yogurt is shown in FIG. 25.

Some thickening agents like starches (modified and non-modified) and hydrocolloids (xanthan gum, carrageenan, guar, locos bean, gellan, etc) can be added to make their texture closer to the real dairy yogurts or Greek yogurts. Strawberry toping, strawberry puree, or toppings or purees from other fruits can be added to improve the flavor. The levels of fruit topping or purees can range from 20%-30%. Small amount of Vanilla extracts, lemon, chocolate or other flavors can also be included in the formulation to improve the flavor.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 62/911,970, filed Oct. 7, 2019, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A food composition for humans comprising Methylococcus capsulatus protein isolate, wherein the Methylococcus capsulatus protein isolate:
(a) is composed of more than 70% by weight crude protein;
(b) has a protein digestibility corrected amino acid score (PDCAAS) of at least 0.9; and
(c) has a δ13C of about −70‰ to about −30‰.

2. The food composition of claim 1, wherein the Methylococcus capsulatus protein isolate is composed of at least 71%, 72%, 73%, 74%, 75%, 76%, 77%, 78%, 79%, 80%, 81%, 82%, 83%, 84%, 85%, 86%, 87%, 88%, 89%, 90% by weight crude protein.

3. The food composition of claim 1 or 2, wherein the Methylococcus capsulatus protein isolate has a δ¹³C of about −60‰ to about −40‰.

4. The food composition of any one of claims 1-3, wherein the Methylococcus capsulatus protein isolate has:
(a) less than 6% by weight nucleic acids;
(b) less than 7% by weight ash;
(c) less than 10% by weight crude fat;
(d) at least 0.05 mg heme/g protein isolate;
or any combination thereof.

5. The food composition of any one of claims 1-4, wherein the Methylococcus capsulatus is not genetically modified.

6. The food composition of any one of claims 1-5, wherein the Methylococcus capsulatus is Methylococcus capsulatus (Bath), Methylococcus capsulatus (Texas), or Methylococcus capsulatus (Aberdeen).

7. The food composition of any one of claims 1-6, wherein the Methylococcus capsulatus protein isolate is derived from Methylococcus capsulatus grown on a carbon feedstock comprising methane or methanol.

8. The food composition of claim 7, wherein the carbon feedstock comprising methane is natural gas or unconventional natural gas.

9. The food composition of any one of claims 1-8, wherein the Methylococcus capsulatus protein isolate is derived from Methylococcus capsulatus grown cultured under low copper conditions.

10. The food composition of claim 9, wherein the Methylococcus capsulatus protein isolate is derived from Methylococcus capsulatus cultured in at most 100 mg copper/kg dry cell weight (DCW).

11. The food composition of claim 9, wherein the Methylococcus capsulatus protein isolate is derived from Methylococcus capsulatus cultured in 1 to 100, from 1 to 10, from 10 to 20, from 20 to 30, from 30 to 40, from 40 to 50, from 50 to 60, from 60 to 70, from 70 to 80, from 80 to 90, from 90 to 100, from 1 to 90, from 1 to 80, from 1 to 70, from 1 to 60, from 1 to 50, from 1 to 40, from 1 to 30, from 10 to 90, from 10 to 80, from 10 to 70, from 10 to 60, from 10 to 50, from 10 to 40, from 10 to 30, from 20 to 90, preferably from 20 to 80, from 20 to 70, from 20 to 60, from 20 to 50, or from 20 to 40 mg copper/kg biomass.

12. The food composition of any one of claims 1-11, wherein isolation of Methylococcus capsulatus protein isolate from Methylococcus capsulatus biomass comprises an acid precipitation step.

13. The food composition of any one of claims 1-12, wherein the Methylococcus capsulatus protein isolate is a liquid, gel, powder, grain, flake, chunk, slurry, nugget, or crumb.

14. The food composition of any one of claims 1-13, wherein the protein isolate is produced by: disrupting Methylococcus capsulatus cells to form a cell lysate, separating and/or concentrating proteins from the cell lysate to form the Methylococcus capsulatus protein isolate, and optionally drying the protein isolate.

15. The food composition of any one of claims 1-14, wherein the Methylococcus capsulatus protein isolate is derived from Methylococcus capsulatus that is cultured and processed under good manufacturing practice (GMP) conditions.

16. The food composition of any one of claims 1-15, wherein the Methylococcus capsulatus protein isolate:
(a) has gelation strength of about 10 g to about 300 g;
(b) is stable as an emulsion during storage at room temperature for at least 24 hours;
(c) exhibits a foam overrun of at least 200%;
(d) has solubility of at least 75% at neutral pH; or
any combination thereof.

17. The food composition of claim 16, wherein the food composition has a gelation strength of about 50 g to about 300 g, and the food composition is a meat-substitute, fish/seafood-substitute, extruded snack, pudding, baked good, pasta, or chip.

18. The food composition of claim 16, wherein the food composition is stable as an emulsion during storage at room temperature for at least 24 hours, and the food composition is a sauce, dip, dressing, soup, vinaigrette, protein shake, nutritional beverage, or dairy substitute.

19. The food composition of claim 16, wherein the food composition has a foam overrun of at least 200%, and the food composition is a meat substitute, chip, egg replacement, or baked good.

20. The food composition of claim 16, wherein the food composition has a solubility of at least 80% at neutral pH, and the food composition is a sauce, dip, dressing, soup, vinaigrette, dairy substitute, protein shake, nutritional beverage, protein gel, or protein supplement powder.

21. The food composition of claim 17 or 19, wherein the meat substitute is a patty, meatball, crumble, sausage, jerky, loaf, filet, bacon, hot dog, or nugget.

22. The food composition of claim 21, wherein the meat substitute is a patty comprising Methylococcus capsulatus protein isolate, a plant-based protein, oil, and optionally water, a binder, at least one spice and/or herb, or a combination thereof.

23. The food composition of claim 18 or 20, wherein the dairy substitute is a non-dairy milk, non-dairy creamer, non-dairy cream, non-dairy yogurt, non-dairy whipped topping, or non-dairy ice cream.

24. The food composition of claim 23, wherein the dairy substitute is a non-dairy ice cream that comprises Methylococcus capsulatus protein isolate, water, sweetener, oil, and optionally fruit, filling, flavoring, or a combination thereof.

25. The food composition of claim 17 or 19, wherein the baked good is a meringue, cracker, cake, pie, pastry, cookie brownie, bun, bread, or quick bread.

26. The food composition of any one of claims 1-25, wherein the food composition further comprises at least one food additive.

27. The food composition of claim 26, wherein the food additive is an emulsifying agent, viscosity increasing agent, binder, anti-caking agent, preservative, sweetener, extract, natural flavoring, flavor enhancer, fat replacement, pH control agent, leavening agent, humectant, nutrient, edible ingredient, oil, or a combination thereof.

28. The food composition of any one of claims 1 to 27, comprising the Methylococcus capsulatus protein isolate in an amount of about 0.5% to about 35% by weight.
